(19) United States
(12) Patent Application Publication
(10) Pub. No.: US 2015/0258042 A1
(43) Pub. Date: Sep. 17, 2015

(54) MODIFIED AND IMMEDIATE RELEASE FORMULATIONS OF MEMANTINE

(71) Applicant: Forest Laboratories Holdings Ltd., Hamilton (BM)

(72) Inventors: Mahendra G. DEDHIYA, Pomona, NY (US); Suneel K. RASTOGI, Ballwin, MO (US); Anil CHHETTRY, Holtsville, NY (US); Narasimhan MANI, Morris Plains, NJ (US); Niranjan RAO, Belle Mead, NJ (US); Antonia PERICLOU, Rutherford, NJ (US)

(21) Appl. No.: 14/722,589

(22) Filed: May 27, 2015

Related U.S. Application Data
(63) Continuation of application No. 13/944,434, filed on Jul. 17, 2013.
Application No. 13/944,434 is a continuation of application No. 13/617,870, filed on Sep. 14, 2012, now abandoned.
Application No. 13/617,870 is a continuation of application No. 13/396,776, filed on Feb. 15, 2012, now abandoned.
Application No. 13/396,776 is a continuation of application No. 11/424,024, filed on Jun. 14, 2006, now abandoned.
(60) Provisional application No. 60/691,512, filed on Jun. 16, 2005.

(51) Int. Cl. A61K 31/13; A61K 9/50; A61K 9/16

(52) U.S. Cl. CPC A61K 31/13; A61K 9/1676; A61K 9/1623; A61K 9/1635; A61K 9/5047; A61K 9/5026; A61K 9/5078

(57) ABSTRACT

The present invention provides immediate release and modified release oral dosage forms. Specifically, the invention provides modified and immediate release pharmaceutical dosage forms containing memantine that exhibit an enhanced release profile and provide reliable absorption. The dosage forms may be used to treat mild, moderate or severe Alzheimer's disease or neuropathic pain.

### CROSS-REFERENCE TO RELATED APPLICATIONS

This application claims priority under 35 U.S.C. §119, to U.S. Provisional Application Ser. No. 60/691,512 filed Jun. 15, 2005, which is hereby incorporated by reference in its entirety.

### FIELD OF THE INVENTION

The present invention is directed to pharmaceutical oral dosage forms that exhibit a modified and/or immediate release profile. The invention is particularly suitable for once a day, oral, pharmaceutical dosage forms in which the active ingredient is memantine, releasing a therapeutically effective amount of memantine over a targeted time period.

### BACKGROUND OF THE INVENTION

Solid oral drug compositions or preparations may be constructed to exhibit various release profiles such as a modified release profile (USP XXV, CDER, FDA, Rockville, Md.), an extended release profile as referenced by FDA Guidelines (“Extended Release Oral Dosage Forms: Development, Evaluation, and Application of In Vitro/In Vivo Correlations”, Food and Drug Administration, CDER, September 1997, Page 17), or an immediate release profile as referenced by FDA guidelines (“Dissolution Testing of Immediate Release Solid Oral Dosage Forms”, issued August 1997, Section IV-A).

In the dissolution testing guideline for modified release profiles, material dissolves over a period of time, and its dissolution is measured at given intervals during this period. A minimum of three time points is recommended and generally cover early, middle and late stages of the dissolution profile. The last measurement should be no earlier than the time point where at least 80% of the drug is dissolved (Guidance for Industry, “Extended Release Oral Dosage Forms: Development, Evaluation, and Application of In Vitro/In Vivo Correlations”, Food and Drug Administration, CDER, September 1997, Page 17). Adequate sampling is important: for example, at 1, 2 and 4 hours and every two hours thereafter until 80% of the drug is released (Guidance for Industry, SUPAC-MR: Modified Release Solid Oral Dosage Forms,” Food and Drug Administration, CDER, September 1997, Page 6). The preferred dissolution apparatus is USP apparatus I (basket) or II (paddle), used at recognized rotation speeds, e.g., 100 rpm for the basket and 50-75 rpm for the paddle (Guidance for Industry, “Extended Release Oral Dosage Forms: Development, Evaluation, and Application of In Vitro/In Vivo Correlations”, Food and Drug Administration, CDER, September 1997, Page 4). Modified release dosage forms permit the release of the active ingredient over an extended period of time in an effort to maintain therapeutically effective plasma levels over similarly extended time intervals, improve dosing compliance, and/or to modify other pharmacokinetic properties of the active ingredient, such as delay onset of release or change conditions under which release occurs.

In the dissolution testing guidelines, materials which dissolve at least 80% in the first 30 to 60 minutes in solution qualify as immediate release profiles. (“Dissolution Testing of Immediate Release Solid Oral Dosage Forms”, issued August 1997, Section IV-A). Therefore, immediate release solid oral dosage forms permit the release of most, or all, of the active ingredient over a short period of time, such as 60 minutes or less, and make rapid absorption of the drug possible.

A multiphase release profile (i.e., a composition containing an immediate release component and at least one modified release component) may be employed to attain one or more combinations of release rates to attain more specific therapeutic objectives such as a portion of drug releasing immediately, followed by an extended release of the remainder. However, modulation of the release rate of an active ingredient does not necessarily ensure that long-lasting effective blood level concentrations will be consistently achieved or that the pharmacological effect will be based solely on the release of the drug, or that pharmacological adverse events will be predictable.

Various formulation techniques have been used to provide a sustained release formulation of soluble drugs. In many such formulations, a drug-containing or drug-bearing particle is coated by one or more release retardant layers or films or is dispersed within a continuous matrix such as a polymeric matrix. The coating layer or the matrix comprises a relatively insoluble material or materials, and the release of the drug is controlled by means of the resistance or permeability of the coating layer or matrix against the diffusion of the drug there through. The release of the drug from such formulations is driven by diffusion into the formulation, e.g., by the gradient of the drug concentration resulting from penetration of, e.g., gastric fluid.

One or more film-forming polymers may be employed to provide sustained release of the active substance by controlling its rate of diffusion across the film barrier(s). However, such an approach may be compromised for tablets if, during ingestion of the oral dosage form, the film is prematurely breached, as by chewing, splitting or abrasion, thereby releasing an excessive amount of active ingredient, which can result in undesirable effects from excessive single-shot drug release, and in failure of the dosage form to remain effective for the required duration. This may be avoided by using, for example, bead formulations that would not be subject to similar mechanical breakage due to their small geometry.

In a matrix-type controlled release approach, lipophilic substances, e.g., higher alcohols, waxes, or insoluble thermoplastic materials, are employed. The release is controlled by the rate of diffusion of the active ingredient into the surrounding medium and, if the matrix itself is degradable, by the rate of its degradation. One of the disadvantages is that a complete release of drug from the matrix tablet is frequently not achieved in practice. Another drawback is that dose proportionality of the dosage forms is not readily achieved, thus, requiring different compositions for different strengths. Thus, the matrix composition to formulate a 5 mg sustained release tablet dosage form may be different from the matrix composition to formulate a 60 mg sustained release tablet dosage form.

U.S. Pat. No. 5,382,601 provides solid pharmaceutical dosage forms containing memantine, which exhibit an extended two-phase release profile, with a portion of the drug being released immediately, followed by a sustained release of the remainder. The matrix of this formulation contains both a water-soluble and a water-insoluble salt of casein, preferably sodium and calcium caseinate. However, casein has an unpleasant taste; it is linked with exacerbation of some side effects as disclosed in U.S. Pat. No. 6,413,556; and displays instability in varying pH. Another concern regarding casein is the possibility of Bovine Spongiform Encephalitis (BSE) contamination since casein is an animal-derived milk protein.

A general method of preparing modified release for N-methyl-D-aspartate (NMDA) receptor antagonists, was described in U.S. Pat. No. 6,194,000. This method involves preparing an immediate release component and a modified release component to arrive at the final formulation. The patent discloses a pellet consisting of a coated core, the coating being any suitable coating using organic solvent-based systems. The patent also does not disclose how the release rates affect the Tₘₐₓ (time to maximum plasma concentration) nor teach how this procedure will result in dose-proportional formulations. U.S. Pat. Nos. 5,382,601 and 6,194,000 describe an extended two-phase release profile incorporating an immediate release component.

Currently, a dosing regimen of memantine twice a day is employed using immediate release tablets. Such a regimen is not optimal because patient compliance decreases as the frequency of taking a drug increases. Moreover, after oral administration, memantine is completely absorbed (absolute bioavailability of approximately 100%). Thus, administration of an immediate-release tablet can lead to greater frequency of adverse pharmacological events due to the fast rate of absorption. Current guidelines for use of memantine in the treatment of Alzheimer's Disease recommends that memantine be administered as a starting dose of 5 mg/day and escalated to the 20 mg/day dose by weekly increases in the dose by 5 mg. Modified release formulations may address some of the concerns associated with the use of memantine.

There is an existing and continual need for a once a day modified and/or immediate release formulation containing memantine, or a pharmaceutically acceptable salt of memantine, with reliable absorption over a targeted period of time. Accordingly, the present invention provides modified and immediate release pharmaceutical dosage forms containing memantine that exhibit an enhanced release profile and provide reliable absorption.

### SUMMARY OF THE INVENTION

According to the present invention, it has now been found that memantine, and its salts, including the hydrochloride salt as well as other of its pharmaceutically acceptable salts can be formulated into a modified release forms with reliable absorption and therefore improved tolerability and an immediate release form with dose-proportional bioavailability.

The present invention provides oral dosage forms that include memantine or a salt thereof, wherein the dosage form comprises 2.5 to 100 mg of memantine or a salt thereof and provides an in vivo plasma profile with a mean Tmax of about 5 or more hours, a mean Cmax of less than about 100 ng/ml and a mean AUC_{0-∞} of more than about 250 ng h/ml. In some embodiments, the oral dosage forms provide a Cmax of less than about 75 ng/ml, preferably less than about 50 ng/ml. In other embodiments, the oral dosage forms provide a mean AUC_{0-∞} of more than about 500 ng h/ml, preferably more than about 1000 ng h/ml and more preferably, more than about 2500 ng h/ml.

According to other embodiments, the present invention provides an oral dosage form comprising 2.5 to 100 mg memantine or a salt thereof wherein the dosage form has a dissolution rate of the active ingredient of about 70% to about 80% within about 4 hours to about 24 hours and a Cₘₐₓ of less than about 100 ng/ml, and wherein the dosage form provides a therapeutic effect over approximately 24 hours when administered to a patient in need thereof and provides a reduced incidence of adverse events.

In some embodiments, the present invention provides oral dosage forms comprising a plurality of beads, wherein each bead includes a core having a diameter from about 1 μm to about 1000 μm and an active ingredient comprising memantine or a salt thereof in the range of about 15 to about 350 mg/g of the dosage form, wherein the dosage forms include less than about 2.5% adduct and has a dissolution rate of the active ingredient of more than about 80% within about the first 60 minutes following entry of the dosage forms into a use environment. In further exemplary embodiments, each bead may also be characterized as comprising an inert core; a mixture of memantine as an active ingredient; and a polymer binder coated on the core.

In exemplary embodiments, such an immediate release oral bead dosage form may comprise a plurality of beads, each bead comprising an inert core having a diameter within a range of from about 1 μm to about 1000 μm; and a mixture of memantine as an active ingredient and a polymer binder coated on said inert core, the dosage form containing memantine with the range of about 15 to about 350 mg/g of said dosage form; said dosage form exhibiting less than about 2.5%; and said dosage form having a dissolution rate of more than about 80% within about the first 60 minutes following entry of the said dosage form into a use environment.

In other embodiments, the present invention provides oral dosage forms comprising a plurality of beads, each bead comprising a core having a diameter from about 1 μm to about 1000 μm, and an active ingredient comprising memantine or a salt thereof in the range of about 15 to about 350 mg/g of the dosage form; and a release modifying polymer layer, wherein the dosage form has a dissolution rate of the active ingredient of about 70% to about 80% within about 4 hours to about 24 hours; and wherein the Cₘₐₓ is less than about 100 ng/ml. In further exemplary embodiments, each bead may also be characterized as comprising an inert core; a mixture of memantine as an active ingredient; and a polymer binder coated on the core.

In exemplary embodiments, such a modified release bead dosage form may comprise a plurality of beads, each bead comprising an inert core having a diameter with the range of from about 1 μm to about 1000 μm; a mixture of memantine as an active ingredient and a polymer binder coated on said inert core, the dosage form containing memantine with the range of about 15 to about 350 mg/g of said dosage form; an intermediate seal coating applied over the memantine-binder coating; and a release modifying polymer layer coated on the seal coating; wherein said dosage form has a dissolution rate of from about 70% to about 80% within about 6 hours to about 12 hours; and wherein the Cₘₐₓ is less than about 60 ng/ml.

In further embodiments, the present invention provides composite dosage forms comprising an immediate release component and a modified release component, wherein the immediate release component comprises a first plurality of beads, each bead comprising a first active ingredient comprising memantine or a salt thereof in the range of about 15 to about 350 mg/g of the dosage form, wherein about 80% of the first active ingredient dissolves within about the first 60 minutes following entry of the dosage form into a use environment; and wherein the modified release component comprises a second plurality of beads, each bead comprising a second active ingredient comprising memantine or a salt thereof in the range of about 15 to about 350 mg/g of the dosage form, wherein about 70% to about 80% of the second active ingredient dissolves within about 4 hours to about 24 hours following entry of the dosage form into the use environment.

### BRIEF DESCRIPTION OF THE DRAWINGS

FIG. 1 shows the dissolution rate for memantine HCl IR beads prepared using the following cellulose binders: hydroxypropyl cellulose, hydroxypropyl methylcellulose, hydroxypropyl methylcellulose (Opadry®, Colorcon, Pa.), and polyvinyl pyrrolidone (povidone). Specifically, FIG. 1 shows dissolution rate stability for memantine HCl IR beads, 171 mg/g, prepared with povidone as a binder. Samples were measured at 40° C., 75% relative humidity, maintained in an induction sealed container with desiccant for three months. Also shown is the dissolution rate stability for memantine HCl IR beads, 157 mg/g, prepared using an HPMC (Opadry®) binder. Dissolution is shown as the percent drug released over time (minutes). The IR beads are stable and can be prepared using different binders.

FIG. 2 shows the dissolution rate stability for memantine HCl MR beads, 142 mg/g, after heating the beads at 50° C. The beads were prepared with HPMC as a binder and with the release modifying polymer coating, Surelease® (Colorcon, Pa.). Dissolution is shown as the percent drug released over time (hours).

FIG. 3 shows the dissolution rate stability for memantine HCl MR beads, 159 mg/g (Release 3) and memantine HCl MR beads, 163 mg/g (Release 1). The beads were prepared with PVP binder, and the release modifying polymer coating was Surelease® (Colorcon, Pa.). Release 3 beads were coated with a 6% w/w weight gain, and the Release 1 beads were coated with a 3% w/w weight gain. Both batches were subsequently coated with an Opadry® top coating. Dissolution is shown as percent dissolved over time (hours).

FIG. 4 shows comparative dissolution rates for memantine HCl IR and MR beads. The MR beads, 163 mg/g (R1), were prepared with PVP, and the modified release polymer was Surelease® at about 3% by weight. Dissolution as percent dissolved, is plotted against time (hours).

FIG. 5 shows the dissolution rate stability for memantine HCl MR beads, 163 mg/g (R1), where immediate release beads were initially prepared with PVP, and a release modifying polymer Surelease® was coated at about 3% by weight based on the IR bead weight. Also shown is memantine HCl modified release beads, 159 mg/g (R3), where immediate release beads were initially prepared with PVP, and a release modifying polymer Surelease® was coated at about 6% based on the weight of the IR beads. Dissolution, as percent dissolved, is plotted against time (hours). Beads were stored at 1 and 3 months at 40° C./75% relative humidity in white HDPE bottles. Data for 3 months are shown.

FIG. 6 shows the dissolution rate stability for the memantine HCl MR beads, 144 mg/g (R4), where immediate release beads were prepared with Hydroxypropyl Methylcellulose (Opadry®) as a binder for drug loading, and the modified release polymers were Ammonio Methacrylate Copolymers—Eudragit® RS and RL at a ratio of 95:5. The total weight gain for Eudragit® was 6% w/w. The beads were stored at 40° C./75% RH in white HDPE bottles. Dissolution is shown as the percent drug dissolved over time (hours). High F2 values (>50) for comparison of initial dissolution rate with that of the stored samples indicated excellent dissolution stability. This formulation was identical to that used in a Pharmacokinetic study. Data for 3 months are shown.

FIG. 7 shows the dissolution rate stability for the memantine HCl beads, 136 mg/g (R5), and memantine HCl beads, 123 mg/g (R6), where immediate release beads were prepared with Hydroxypropyl Methylcellulose (Opadry®) as a binder for drug loading, and the modified release polymers were Ammonio Methacrylate Copolymers—Eudragit® RS and RL at ratio of 95:5. The total weight gain for Eudragit® was 10% w/w and 20% w/w for R5 and R6 beads respectively. The beads were stored at 40° C./75% RH in white HDPE bottles. Dissolution is shown as the percent drug dissolved over time (hours). High F2 values (>50) for comparison of initial dissolution rate with that of the stored samples indicated excellent dissolution stability. Data for 3 months are shown.

FIG. 8 shows the dissolution rates for modified release beads R3 (Bio formula) formulated at 25, 40 and 60 mg dose strengths for the purpose of testing whether dose proportionality is achieved. Dissolution is shown as percent drug dissolved over time (hours). The memantine HCl beads, 159 mg/g (R3), were prepared using Povidone as a binder for drug loading, and the release modifying polymer was ethylcellulose (Surelease®).

FIG. 9 shows the dissolution profiles for formulations comprising a plurality of beads (R1 and R3) in various ratios. As used herein the ratio may refer to either the amount of beads (e.g., 2:38 may mean 2 mg R1 and 38 mg of R3) or % of beads (e.g., 5:95 may mean 5% R1 and 95% R3). The Memantine HCl Release 1 beads, 163 mg/g, and Memantine HCl Release 3 beads, 159 mg/g were prepared using Povidone as a binder for drug loading and the release modifying polymer was Ethylcellulose (Surelease®). Also shown is the dissolution profiles for formulations comprising a plurality of beads (R4, R5 and R6) in various ratios. The data show that by combining different ratios of beads having different release characteristics, similar dissolution profiles can nevertheless be achieved. The Memantine HCl Release 4 beads, 144 mg/g, Memantine HCl Release 5 beads, 136 mg/g and Memantine HCl Release 6 beads, 123 mg/g were prepared using Hydroxypropyl Methylcellulose (Opadry®) as a binder for drug loading and release modifying polymers were ammonio methacrylate copolymers—Eudragit® RS and RL at a ratio of 95:5. Dissolution is shown as percent drug dissolved over time (hours).

FIG. 10 shows the dissolution rate stability for a capsule dosage form for memantine HCl MR beads, 40 mg, comprising two types of beads (R1:R3 ratio of 2 mg:38 mg) and a capsule dosage, 40 mg, form composed of the memantine HCl MR beads comprising two types of beads (R1 and R3). The stability study was conducted by dissolution tests after storing the beads at 25° C./60% relative humidity in white HDPE bottles. Also shown is the dissolution rate stability for a capsule dosage form composed of the memantine HCl MR beads comprising a combination of beads R4, R5 and R6. The ratio (%) of Release 4:Release 5:Release 6 beads was 8:35:57, and the combined dose strength was 40 mg. The stability study was conducted by dissolving after storing the beads at 25° C./60% relative humidity in white HDPE bottles. Dissolution is shown as the percent drug dissolved over time (hours).

FIG. 11A shows the blood plasma concentrations values of memantine from Treatments A, B, C and D. The memantine plasma concentration profile in ng/ml is plotted over time (hours). FIG. 11B shows a portion of the memantine plasma concentration profiles of FIG. 11A plotted over a truncated timeline (hours) and an expanded abscissa scale compared to FIG. 11B.

FIG. 12 shows the dissolution profiles for a 40 mg capsule, comprising a plurality of beads, (R1:R3, ratio of 10 mg:30 mg) or (R1:R3 ratio of 2 mg:38 mg), in different biorelevant dissolution media representing different pH values [fed (FeSSIF) and fasted (FaSSIF) state simulated intestinal fluid]. These plots indicate that the dissolution profiles are pH independent. Dissolution, percent drug dissolved, is plotted against time (hours).

FIG. 13 shows the dissolution rate for memantine HCl MR beads, 92 mg/g, under two different pH conditions: 1.2 and 5.6. The immediate release beads were prepared with Hydroxypropyl Methylcellulose (Opadry®) as a binder for drug loading, and the release modifying polymer was Methacrylic Acid Copolymer Type C, NF (Acryl-Eze®). The total weight gain for Acryl-Eze® was 30% w/w. The stability study was conducted after storing the beads at 40° C./75% relative humidity in white HDPE bottles. Dissolution, percent drug dissolved, is plotted against time (hours).

### DETAILED DESCRIPTION OF THE INVENTION

In accordance with the present invention, oral dosage forms are provided for administration of memantine, or one of its pharmaceutically acceptable salts, preferably its HCl salt, to a human, where the composition includes memantine in solid oral dosage forms. In particular, the pharmaceutical compositions of the present invention are directed to immediate and/or modified release compositions of memantine, or one of its pharmaceutically acceptable salts.

Immediate and modified release formulations of memantine have been disclosed in U.S. application Ser. No. 11/155,319 (Published as US2006/0002999) and U.S. application Ser. No. 11/155,330 (Published as US2006/0051416), the disclosures of which are hereby incorporated by reference in their entirety.

The present invention provides oral dosage forms that include memantine or a salt thereof, wherein the dosage form comprises 2.5 to 100 mg of memantine or a salt thereof and provides an in vivo plasma profile with a mean Tmax of about 8 or more hours, a mean Cmax of less than about 100 ng/ml and a mean AUC_{0-∞} of more than about 250 ng h/ml.

According to some embodiments, the present invention provides an oral dosage form comprising memantine or a salt thereof, wherein the dosage form comprises 2.5 to 50 mg of memantine or a salt thereof and provides an in vivo plasma profile with a mean Tmax of about 5 or more hours, a mean Cmax of less than about 50 ng/ml and a mean AUC_{0-∞} of more than about 250 ng h/ml.

In some embodiments, the oral dosage forms provide a Cmax of less than about 75 ng/ml, preferably less than about 50 ng/ml. In other embodiments, the oral dosage forms provide a mean AUC_{0-∞} of more than about 500 ng h/ml, preferably more than about 1000 ng h/ml.

According to other embodiments, the present invention provides an oral dosage form comprising 2.5 to 100 mg memantine or a salt thereof wherein the dosage form has a dissolution rate of the active ingredient of about 70% to about 80% within about 4 hours to about 24 hours and a Cₘₐₓ of less than about 100 ng/ml, and wherein the dosage form provides a reduced incidence of adverse events.

Memantine (1-amino-3,5-dimethyladamantane), which is an analog of 1-amino-cyclohexane (disclosed, e.g., U.S. Pat. Nos. 4,122,193; 4,273,774; 5,061,703), is a systemically-active uncompetitive NMDA receptor antagonist having low to moderate affinity for the receptor and strong voltage dependency and rapid blocking/unblocking kinetics. These pharmacological features allow memantine to block sustained activation of the receptor under pathological conditions and to rapidly leave the NMDA channel during normal physiological activation of the channel. Memantine, and pharmaceutically acceptable salts thereof (e.g., the HCl salt, MW 215.77), is approved in the U.S. for treatment of Alzheimer's disease. Approval of memantine is currently sought for the indication of neuropathic pain (wherein memantine has demonstrated activity in in vitro models), and is currently approved outside the United States as an oral formulation for both Alzheimer's and Parkinson's Disease.

According to the invention, memantine may preferably be used in the form of a pharmaceutically acceptable salt. Suitable salts of the compound include, but are not limited to, acid addition salts, such as those made with hydrochloric, methylsulfonic, hydrobromic, hydroiodic, perchloric, sulfuric, nitric, phosphoric, acetic, propionic, glycolic, lactic pyruvic, malonic, succinic, maleic, fumaric, maleic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. In a preferred embodiment, the salt is memantine hydrochloride (C₁₂H₂₁N.HCl, MW 215.77). The term “salts” can also include addition salts of free acids or free bases. All of these salts (or other similar salts) may be prepared by conventional means. All such salts are acceptable provided that they are non-toxic and do not substantially interfere with the desired pharmacological activity.

In addition, it is possible to use any salts and free base form of memantine including polymorphs, hydrates and solvates as well as amorphous forms of memantine. As used below in the present specification and claims “memantine” will be deemed to encompass both the free base and pharmaceutically acceptable salts thereof. In preferred embodiments of the invention, the active ingredient is memantine hydrochloride.

In some embodiments, the present invention provides oral dosage forms comprising a plurality of beads, wherein each bead includes a core having a diameter from about 1 μm to about 1000 μm and the core includes an active ingredient comprising memantine or a salt thereof in the range of about 15 to about 350 mg/g of the dosage form, wherein the dosage forms include less than about 2.5% adduct and has a dissolution rate of the active ingredient of more than about 80% within about the first 60 minutes following entry of the dosage forms into a use environment. In preferred embodiments, the dissolution rate is more than about 80% within 30 minutes.

In some embodiments of the present invention, the oral dosage forms include a plurality of beads, wherein each bead includes a core and an active ingredient comprising memantine. A suitable IR bead form of memantine may simply be particles of memantine admixed with soluble components for example, sugars (e.g., sucrose, mannitol, etc.), polymers (e.g., polyethylene glycol, hydroxypropyl cellulose, hydroxypropyl methyl cellulose, etc.), surfactants (sodium lauryl sulphate, chremophor, tweens, spans, pluronics, and the like), insoluble glidant components (microcrystalline cellulose, calcium phosphate, talc, fumed silica, and the like), coating material (examples of suitable coating materials are polyethylene glycol, hydroxypropyl methyl cellulose, wax, fatty acids, etc.), dispersions in suitable material (examples are wax, polymers, pharmaceutically acceptable oils, soluble agents, etc.) or combinations of the above.

According to some embodiments, the core contemplated in the invention include, but are not limited to, sugar spheres (nonpareil seeds), microcrystalline cellulose, or mannitol. Preferably, the core is a sugar sphere, USP (Paulaur Cranbury, N.J.). The particle size of the core ranges from about 1 μm to about 1000 μm, preferably in the range of about 300 μm to about 900 μm, and more preferably within the range of from about 450 μm to about 825 μm. In exemplary embodiments, the core may be coated to avoid interaction between the core and the active ingredient. For example, suitable coating materials include, but are not limited to, polyethylene glycol, hydroxypropyl methyl cellulose, wax, fatty acids, etc.

In one embodiment, the spheres comprise a portion of the dosage form ranging from about 100 mg/g to about 950 mg/g, preferably from about 550 mg/g to about 850 mg/g. In another embodiment, the spheres comprise a portion of the dosage form ranging from 620 mg/g to about 930 mg/g, preferably from about 700 mg/g to about 850 mg/g. The fraction of the bead will depend on the amount of additional constituents, if any, used in the dosage form.

The core is coated with memantine, preferably memantine hydrochloride. In one embodiment, memantine HCl is present in amounts from about 15 mg/g to about 350 mg/g, preferably from about 50 to 300 mg/g based on the weight of the entire IR bead. In other embodiments, memantine is present in amounts from about 15 to 300 mg/g, preferably from about 25 to about 250 mg/g.

In a preferred embodiment, the memantine hydrochloride is added to a mixture of a binder and a glidant prior to coating the core with the memantine. The glidant may be selected from, but is not limited to, microcrystalline cellulose, calcium phosphate, talc, fumed silica. Glidants may be used in amounts ranging from 1.5 mg/g to about 35 mg/g, preferably from about 1.5 mg/g to about 30 mg/g, more preferably from about 2.5 mg/g to about 25 mg/g. In another embodiment, the preferred range of glidant is from about 5 mg/g to about 30 mg/g.

The binder may be selected from, but is not limited to, povidone (PVP), hydroxypropyl methylcellulose (HPMC, Opadry), hydroxypropyl cellulose (HPC), or combinations thereof. In an embodiment where the binder is HPMC, the binder is present in an amount ranging from about 15 mg/g to about 30 mg/g, preferably from about 15 mg/g to about 25 mg/g. In another embodiment, where the binder is povidone, the binder is present in an amount of from about 1.5 mg/g to about 35 mg/g, preferably from about 5 mg/g to about 30 mg/g.

The following table provides one exemplary embodiment of the invention.

TABLE 1

| | | Range | Preferred |
|---|---|---|---|
| Ingredients | | (mg/g) | range (mg/g) |
| Active Ingredient (AI) | Memantine HCl | 15-350 | 50-300 |
| Binder | Povidone, USP | 1.5-35 | 5-30 |
| Glidant | Talc, USP | 1.5-35 | 5-30 |
| Core | Sugar Spheres, USP | 620-930 | 700-850 |
| | Or Microcrystalline | | |
| | Cellulose Spheres | | |

| | | |
|---|---|---|
| Water | Purified Water, USP | Purified Water is removed |
| | | during the process |

| | |
|---|---|
| Total (Drug Loaded Beads) | 1000 |

The mixture of active ingredient and binder/water/glidant may be prepared by mixing, e.g., with a stirrer, for at least 15 minutes, preferably at least 30 minutes, more preferably at least one hour. The components may also be combined by methods including blending, mixing, dissolution and evaporation, or by using suspensions.

The active ingredient/binder/inactives mixture may be deposited on a core, wet massed and extruded, granulated, or spray dried. In one embodiment, sugar spheres are prewarmed to a temperature ranging from about 40° C. to about 55° C. prior to application of the mixture. The core may be optionally coated with from about 2% w/w to about 10% w/w seal coating prior to applying the active drug layer. The seal coating may be any applicable coating which can separate any active ingredients from the core, for example, polymer coatings such as Eudragit®, HPMC, HPC, or combinations thereof. For this reason also, dissolution stability (i.e., maintenance of dissolution profile after exposure to elevated temperatures) is important for the compositions of the present invention.

In one embodiment, the sugar sphere are coated with a fluidized bed coater known in the art, for example, a Glatt Powder Coater and Granulator, GPCG3 (Ramsey, N.Y.). One skilled in Coating conditions such as air velocity, spray rate, and atomization pressure are typically controlled as is appreciated by and known to those skilled in the art. The temperature range of the product may range from about 43° C. to about 51° C. The air velocity may range from about 5 to about 9 m/s. The spray rate ranges from about 9 to about 42 gm/min. The atomization pressure preferably ranges from about 1.5 to about 2.0 bar. The beads are then dried in the fluidized bed of the coating apparatus at a temperature of about 45° C. to about 50° C. for at least 5 minutes, preferably at least 15 minutes, more preferably at least 30 minutes. One skilled in the art will recognize that many alternate operating conditions and various types of equipment can also be used.

Once the IR beads are formed as cores containing coated drug, the beads may be optionally additionally coated with a seal coating. The seal coating may be a polymer or a combination of polymers that can be designed to be pH dependent or independent. In a preferred embodiment, the polymer for the seal coating is selected from, but are not limited to HPMC (Opadry®, Colorcon, Pa.), HPC, Eudragit® RL, Eudragit® E100, Eudragit® E 12.5, Eudragit®, E PO, Eudragit® NE (e.g., NE 30D or NE 40D) and combinations of two or more of the foregoing. These polymers are insoluble in aqueous media but display pH-independent swelling on contact with aqueous fluids. In another embodiment, the IR beads are coated with pH-dependent polymers, soluble at a pH preferably above 5. In the IR bead formulations, the seal coating polymer is present in amounts ranging from about 0% w/w to about 40% w/w, preferably from about 0% w/w to about 10% w/w/, more preferably from about 0% w/w to about 3% w/w.

Alternatively the IR cores may be coated with a rapidly disintegrating or dissolving coat for aesthetic, handling, or stability purposes. Suitable materials are polyvinylpyrrolidone, hydroxypropyl cellulose, hydroxypropyl methyl cellulose, polyethylene glycol, polymethacrylates containing free amino groups, each may be with or without plasticizers, and with or without an antitack agent or filler. An addition of about 3% of the weight of the core as coating material is generally regarded as providing a continuous coat for this size range.

The following table (Table 2) demonstrates an exemplary embodiment of the invention with the components used in a coated bead formulation.

TABLE 2

| | | Range | Preferred |
|---|---|---|---|
| Ingredients | | (mg/g) | range (mg/g) |
| Core with drug | Memantine HCl | 15-500 | 25-400 |
| Coating | Hydroxypropyl Methyl- | 0-30 | 0-25 |
| | cellulose (Opadry ®) | | |

| | | |
|---|---|---|
| Water | Purified Water, USP | Purified Water is removed |
| | | during the process |

| | |
|---|---|
| Total (Seal Coated Beads) | 1000 |

In other embodiments, the present invention provides an oral dosage form comprising a plurality of beads, each bead comprising a core having a diameter from about 1 μm to about 1000 μm, wherein the core comprises an active ingredient comprising memantine or a salt thereof in the range of about 15 to about 350 mg/g of the dosage form; and a release modifying polymer layer, wherein the dosage form has a dissolution rate of the active ingredient of about 70% to about 80% within about 4 hours to about 24 hours; and wherein the Cₘₐₓ is less than about 100 ng/ml.

The modified release (MR) beads of the present invention may be prepared initially as IR beads as described above, with a core, layer of active ingredient, and a seal coating. The IR beads may then be coated with an MR component in the form of a release modifying polymer dispersion and preferably an additional topcoat of polymer for aesthetic, handling or stability purposes. The final dosage form, such as a capsule, may contain a different amount of beads depending on the desired dose of the composition.

The polymer dispersion is prepared by mixing water with a polymer selected from, but not limited to, ethylcellulose (Surelease®, Colorcon, Pa.), methacrylate (Eudragit®, Rohm Pharma, NJ), and methacrylic acid copolymer type C (Acryl-eze®, Indianapolis, Ind.). In one embodiment, the dispersion is mixed for at least 15 minutes, preferably at least 30 minutes.

Since binders and matrix polymers have different dissolution stability, the binder and polymer compositions are selected in particular combinations to reduce or eliminate dissolution instability. Depending on which binder is used, particular polymer dispersions are preferred. In one embodiment, where the binder is povidone, the polymer coating is ethylcellulose. In another embodiment, where the binder is HPMC, the polymer is methacrylate or methacrylic acid. Where methacrylate is used as a polymer, triethyl citrate is added to the polymer. After application with a fluidizer, the beads are once again dried. Specific amounts of the various components are disclosed in Tables 3-7.

A final over coating (or top coat) is preferably layered onto the beads or pellets. The over coating may be a polymer selected from, but are not limited to HPMC (Opadry®, Colorcon, Pa.), HPC, Eudragit® RL, Eudragit® E100, Eudragit® E 12.5, Eudragit® E PO, Eudragit® NE and mixtures thereof. Tables 3-7 demonstrate exemplary embodiments of the invention.

TABLE 3
MR beads with Surelease ® 3%/with PVP

| | | Range | Preferred |
|---|---|---|---|
| Ingredients | | (mg/g) | range(mg/g) |
| Active Ingre- | Memantine HCl | 14-350 | 50-285 |
| dient (AI) | | | |
| Binder for AI | Povidone, USP | 1.5-35 | 5-30 |
| Glidant | Talc, USP | 1.5-35 | 5-30 |
| Core | Sugar Spheres, USP | 600-890 | 650-800 |
| | Or Microcrystalline | | |
| | Cellulose Spheres | | |
| Modified Release | Ethylcellulose | 110-120 | 110-120 |
| Polymer** | (Surelease ®) | | |
| Top Coat | Hydroxypropyl | 15-30 | 15-25 |
| | Methylcellulose | | |
| | (Opadry ®) | | |
| Water | Purified Water, USP* | | |
| | Total** | 1000 | |

*Purified Water is removed during the process
**Contains 25% w/w solids

TABLE 4a
MR beads with Surelease ® 6%/with PVP

| | | Range | Preferred |
|---|---|---|---|
| Ingredients | | (mg/g) | range(mg/g) |
| Active Ingre- | Memantine HCl | 15-325 | 30-280 |
| dient (AI) | | | |
| Binder for AI | Povidone, USP | 1.5-32 | 3-28 |
| Glidant | Talc, USP | 1.5-32 | 3-28 |
| Core | Sugar Spheres, USP | 580-850 | 625-780 |
| | Or Microcrystalline | | |
| | Cellulose Spheres | | |
| Modified Release | Ethylcellulose | 212-232 | 212-232 |
| Polymer** | (Surelease ®) | | |
| Top Coat | HPMC (Opadry ®) | 15-30 | 15-25 |
| Water | Purified Water, USP* | | |
| | Total** | 1000 | |

*Purified Water is removed during the process
**Contains 25% w/w solids

TABLE 4b
MR beads with Surelease ® 7%/with PVP

| | | Range | Preferred |
|---|---|---|---|
| Ingredients | | (mg/g) | range(mg/g) |
| Active Ingre- | Memantine HCl | 15-325 | 30-280 |
| dient (AI) | | | |
| Binder for AI | Povidone, USP | 1.5-32 | 3-28 |
| Glidant | Talc, USP | 1.5-32 | 3-28 |
| Core | Sugar Spheres, USP | 580-850 | 625-780 |
| | Or Microcrystalline | | |
| | Cellulose Spheres | | |
| Modified Release | Ethylcellulose | 231-283 | 231-283 |
| Polymer | (Surelease ®) | | |
| Top Coat | HPMC (Opadry ®) | 15-30 | 15-25 |
| Water | Purified Water, USP* | | |
| | Total** | 1000 | |

*Purified Water is removed during the process
**Contains 25% w/w solids

TABLE 4c
MR beads with Surelease ® 10%/with PVP

| | | Range | Preferred |
|---|---|---|---|
| Ingredients | | (mg/g) | range(mg/g) |
| Active Ingre- | Memantine HCl | 15-325 | 30-280 |
| dient (AI) | | | |
| Binder for AI | Povidone, USP | 1.5-32 | 3-28 |
| Glidant | Talc, USP | 1.5-32 | 3-28 |
| Core | Sugar Spheres, USP | 580-850 | 625-780 |
| | Or Microcrystalline | | |
| | Cellulose Spheres | | |
| Modified Release | Ethylcellulose | 350-430 | 350-430 |
| Polymer | (Surelease ®) | | |
| Top Coat | HPMC (Opadry ®) | 15-30 | 15-25 |
| Water | Purified Water, USP* | | |
| | Total** | 1000 | |

*Purified Water is removed during the process
**Contains 25% w/w solids

TABLE 4d
MR beads with Surelease ® 8%/with PVP

| | | Range | Preferred |
|---|---|---|---|
| Ingredients | | (mg/g) | range(mg/g) |
| Active Ingre- | Memantine HCl | 15-325 | 30-280 |
| dient (AI) | | | |
| Binder for AI | Povidone, USP | 1.5-32 | 3-28 |
| Glidant | Talc, USP | 1.5-32 | 3-28 |
| Core | Sugar Spheres, USP | 580-850 | 625-780 |
| | Or Microcrystalline | | |
| | Cellulose Spheres | | |
| Modified Release | Ethylcellulose | 279-340 | 279-340 |
| Polymer | (Surelease ®) | | |
| Top Coat | HPMC (Opadry ®) | 15-30 | 15-25 |
| Water | Purified Water, USP* | | |
| | Total** | 1000 | |

*Purified Water is removed during the process
**Contains 25% w/w solids

TABLE 4e
MR beads with Surelease ® 9%/with PVP

| | | Range | Preferred |
|---|---|---|---|
| Ingredients | | (mg/g) | range(mg/g) |
| Active Ingre- | Memantine HCl | 15-325 | 30-280 |
| dient (AI) | | | |
| Binder for AI | Povidone, USP | 1.5-32 | 3-28 |
| Glidant | Talc, USP | 1.5-32 | 3-28 |
| Core | Sugar Spheres, USP | 580-850 | 625-780 |
| | Or Microcrystalline | | |
| | Cellulose Spheres | | |
| Modified Release | Ethylcellulose | 315-390 | 315-390 |
| Polymer | (Surelease ®) | | |
| Top Coat | HPMC (Opadry ®) | 15-30 | 15-25 |
| Water | Purified Water, USP* | | |
| | Total** | 1000 | |

*Purified Water is removed during the process
**Contains 25% w/w solids

TABLE 5
MR Beads with Eudragit 6% w/w/HPMC

| | | Range | Preferred |
|---|---|---|---|
| Ingredients | | (mg/g) | range(mg/g) |
| Active Ingre- | Memantine HCl | 15-300 | 50-250 |
| dient (AI) | | | |
| Binder for AI | Opadry ® Clear | 15-300 | 50-250 |
| Glidant | Talc, USP | 1-30 | 5-20 |
| Core | Sugar Spheres, USP | 400-750 | 450-700 |
| | Or Microcrystalline | | |
| | Cellulose Spheres | | |
| Coating | Opadry ® Clear | 10-30 | 12-25 |
| Modified Release | Ammonio Methacrylate | 75-100 | 83-93 |
| Polymer | Copolymer NF | | |
| | (Eudragit ®)* | | |
| Modified Release | Ammonio Methacrylate | 0.1-11 | 1-7 |
| Polymer | Copolymer NF Type A, | | |
| | (Eudragit ®)* | | |
| Additive to | Triethyl Citrate, NF | 2-15 | 3-11 |
| Modified | | | |
| Release Polymer | | | |
| Glidant | Talc, USP | 1-40 | 15-35 |
| Top Coating | Opadry ® Clear | 10-30 | 12-25 |
| Water | Purified Water, USP** | | |
| | Total (Seal Coated | 1000 | |
| | Beads) | | |

*Contains 30% w/w solids
**Purified Water is removed during the process

TABLE 6
MR Beads Eudragit 10% w/w/HPMC

| | | Range | Preferred |
|---|---|---|---|
| Ingredients | | (mg/g) | Range (mg/g) |
| Active Ingre- | Memantine HCl | 15-300 | 50-250 |
| dient (AI) | | | |
| Binder for AI | Opadry ® Clear | 15-300 | 50-250 |
| Glidant | Talc, USP | 1-30 | 5-20 |
| Core | Sugar Spheres, USP | 400-750 | 450-700 |
| | Or Microcrystalline | | |
| | Cellulose Spheres | | |
| Coating | Opadry ® Clear | 10-30 | 12-25 |
| Modified Release | Ammonio Methacrylate | 131-175 | 145-162 |
| Polymer | Copolymer NF | | |
| | (Eudragit ®)* | | |
| Modified Release | Ammonio Methacrylate | 1-26 | 7-17 |
| Polymer | Copolymer NF Type A, | | |
| | (Eudragit ®)* | | |
| Additive to | Triethyl Citrate, NF | 3-26 | 5-19 |
| Modified | | | |
| Release Polymer | | | |
| Glidant | Talc, USP | 30-70 | 40-60 |
| Top Coating | Opadry ® Clear | 10-30 | 12-25 |
| Water | Purified Water, USP¹ | | |
| | Total (Seal Coated | 1000 | |
| | Beads) | | |

*Contains 30% w/w solids
¹Purified Water is removed during the process

TABLE 7
MR Beads Eudragit 20% w/w/HPMC

| | | Range | Preferred |
|---|---|---|---|
| Ingredients | | (mg/g) | Range (mg/g) |
| Active Ingre- | Memantine HCl | 15-300 | 50-250 |
| dient (AI) | | | |
| Binder for AI | Opadry ® Clear | 15-300 | 50-250 |
| Glidant | Talc, USP | 1-30 | 5-20 |
| Core | Sugar Spheres, USP | 400-750 | 450-700 |
| | Or Microcrystalline | | |
| | Cellulose Spheres | | |
| Coating | Opadry ® Clear | 10-30 | 12-25 |
| Modified Release | Ammonio Methacrylate | 235-314 | 260-292 |
| Polymer | Copolymer NF | | |
| | (Eudragit ®)* | | |
| Modified Release | Ammonio Methacrylate | 5-48 | 12-31 |
| Polymer | Copolymer NF Type | | |
| | A, (Eudragit ®)* | | |
| Additive to | Triethyl Citrate, NF | 6-47 | 9-35 |
| Modified | | | |
| Release Polymer | | | |
| Glidant | Talc, USP | 50-120 | 65-100 |
| Top Coating | Opadry ® Clear | 10-30 | 12.25 |
| Water | Purified Water, USP** | | |
| | Total (Seal Coated | 1000 | |
| | Beads) | | |

*Contains 30% w/w solids
**Purified Water is removed during the process

TABLE 8
Reference IR Tablet formulation

| Ingredients | (mg/Tablet) |
|---|---|
| Memantine HCl | 20.00 |
| Lactose Monohydrate, NF | 349.50 |
| (FF-Modified Spray Dried) | |
| Microcrystalline Cellulose NF | 104.20 |
| (Avicel ® PH 101) | |
| Colloidal Silicon Dioxide, NF | 2.50 |
| (Cab-O-Sil ® M5) | |
| Talc, USP | 22.30 |
| Magnesium Stearate, NF | 1.50 |
| Core Tablet Weight | 500.00 |
| Opadry ® II White (Y-22-7719) | 15.00 |
| Purified Water, USP | — |
| Coated Tablet Weight | 515.00 |

*Purified water is removed during the process
**Twice the amount required, tablets would be coated to approximately 3.0% weight gain

Drug dissolution from the MR beads occurs by the penetration of the bulk medium and drug diffusion across the polymer layer, which are in turn controlled by the permeability and swelling properties of the polymer. The modified release beads have essentially bioequivalent AUC as compared to an immediate release tablet dosage form, and a reduced Cₘₐₓ of at least 25% relative to the immediate release tablet (Table 8). The modified release bead demonstrates good tolerability and can be administered over a wide range of dosages. Cₘₐₓ (maximum plasma concentration) is less than about 85% of the immediate release tablets when administered as a single dose. AUC (area under the curve, a measure of bioavailability) is within 75% to 130% of the immediate release tablets administered as a single dose. This range is considered equivalent with respect to overall systemic exposure.

All of the beads from the modified release formulation do not release immediately. This is important to prevent dose dumping and to reduce adverse events. In the modified bead formulation, average Tₘₐₓ (time to reach maximum plasma concentration) ranges from between about 5 to about 48 hours, preferably from about 5 to about 36 hours. The beads have an in vitro release rate of more than about 70% to about 80% in about 4 to about 12 hours. Preferably, the formulations have a release rate of about 30% to about 60% in about 2 to about 6 hours. More preferably, the formulations have a release rate of about 10% to about 50% within the first hour following entry into a use environment followed by extended release; more preferably, the formulations have a release rate of about 10% to about 35% within the first hour.

In other embodiments, the present invention provides a composite dosage form comprising an immediate release component and a modified release component, wherein the immediate release component comprises a first plurality of beads, each bead comprising a first active ingredient comprising memantine or a salt thereof in the range of about 15 to about 350 mg/g of the dosage form, wherein about 80% of the first active ingredient dissolves within about the first 60 minutes following entry of the dosage form into a use environment; and wherein the modified release component comprises a second plurality of beads, each bead comprising a second active ingredient comprising memantine or a salt thereof in the range of about 15 to about 350 mg/g of the dosage form, wherein about 70% to about 80% of the second active ingredient dissolves within about 4 hours to about 24 hours following entry of the dosage form into the use environment.

The composite dosage form may be combined into a single dosage form having a uni-phase or multi-phase profile. The active ingredient, e.g., memantine hydrochloride, in the composition may be present in amounts measured as mg per dose, ranging from about 2.5 mg to about 100 mg per dose. Preferably, the doses contain 2.5 mg to 80 mg active ingredient. In other embodiments, the dose is 3, 6, 7, 9, 12, 14, 15, 20, 21, 28, 40 or 60 mg.

The compositions including an IR and MR component may include an amount of memantine in the immediate release form of approximately 5% to 90% of the composition of the invention, preferably 10% to 60%. An immediate release memantine content of about 15% to 50% is particularly preferred. The controlled release form of the memantine may constitute the remainder of the active ingredient. As a result, a final composition provides an amount of memantine for immediate release following administration and an additional amount for sustained/modified release. The composition of the invention may exhibit more than one peak in the plasma concentration/time curve in any one dosing interval depending on a particular active ingredient used, relative amounts of the IR and MR components, and the dissolution properties of the MR component. Thus, compositions may be achieved that have specific release profiles.

The compositions including an IR and MR component may include any solid oral dosage forms known in the art. Preferred solid dosage forms used in the present invention include beads. Beads are dose proportional, i.e., the same proportions of beads of different types can be used for different doses without significantly altering the percent drug released over time. For example, a 40 mg dose will deliver twice the drug as a 20 mg dose, with proportional bioavailability. Different doses are obtained by using different amounts of beads. Beads also enable a variety of dissolution profiles by mixing one or more types of beads with different dissolution properties or using multi-layer coatings, as additional drug layering over a polymer layer and subsequent coatings to prepare unitary beads, as familiar to one skilled in the art. Beads also enable a wide range of drug loading. For example, memantine beads may be loaded on beads at up to 500 mg/g dosage form. One skilled in the art will recognize that higher drug loading allows for smaller capsule size.

Prolonging the time to maximum plasma concentration (Tₘₐₓ) as compared to immediate release tablet, is related to the release rate of the drug in the use environment. The release rate of the drug depends on many factors, including the composition of the solid dosage forms and the dissolution properties. By using different compositions containing either unitary beads or a combination of a plurality of bead types, their individual release rates can be combined to achieve desired plasma release profiles. Beads with different release characteristics can be achieved by selection of the release-modifying polymer, as well as the combination of the release-modifying polymer and the binder to impart different release characteristics to the resulting beds. Overcoats such as enteric coatings can also be used, if desired.

The beads or bead mixtures may be used, for example, in suspensions, filled into capsules, compressed into tablets, or filled into sachets. One or more types of modified release beads can be mixed together and encapsulated, or used as a sprinkle on the subject's food. According to the invention, the oral solid dosage form may be any of these forms. Preferably, the dosage form is a capsule.

In one embodiment of the invention, the beads are formulated into capsules with the use of an encapsulation machine. Various capsule sizes may be required to accommodate the strength and fill weight of the target formulations. Capsule size range from 00 to 5 for fill weights ranging from about 15 mg to about 630 mg.

The particle sizes of the IR and MR bead components in the dosage form depend on the technology used to prepare them. The particle sizes component range from submicron to 500 μm for powder technologies (mixtures, spray drying, dispersions etc), 5 to 1700 μm for coating technologies (Wurster®, top spray, bottom spray, spray drying, extrusion, layering, etc.), to 1-40 mm for tabletting technologies.

In accordance with the present invention, oral dosage forms are provided for administration of memantine, or one of its pharmaceutically acceptable salts, preferably its HCl salt, to a human. The oral dosage forms of the invention are suitable for the treatment of CNS disorders, including but not limited to the treatment of Alzheimer's disease, Parkinson's disease, AIDS dementia (U.S. Pat. Nos. 5,506,231, 5,061,703, and 5,614,560; see also Parsons et al., Neuropharmacology 1999 June; 38(6):735-67), neuropathic pain (U.S. Pat. No. 5,334,618), cerebral ischemia (U.S. Pat. No. 5,061,703), epilepsy, glaucoma, hepatic encephalopathy, multiple sclerosis, stroke, depression (U.S. Pat. No. 6,479,553), tardive dyskinesia, malaria, Boma virus, Hepatitis C (U.S. Pat. Nos. 6,034,134 and 6,071,966). Additional pathologies for treatment of which memantine is suitable are disclosed in U.S. Pat. Nos. 5,614,560 and 6,444,702. Of particular interest is the ability to provide uninterrupted pain relief. Accordingly, the present invention further provides a method for the therapeutic or prophylactic treatment of CNS disorders in a human or animal subject, the method including administering to the subject in need of such treatment, a composition in accordance with the present invention in an amount effective to treat the CNS disorder.

### DEFINITIONS

For purposes of the present invention, “sustained release” or modified release” means that the release of the therapeutically active agent occur over an extended period of time leading to lower peak plasma concentrations and/or is directed to a prolonged Tₘₐₓ as compared to “immediate release.” For example, modified release compositions may have a mean Tₘₐₓ of about 5 or more hours.

The term “dissolution requirement” means the dissolution rate of beads obtained when tested using the equipment and procedure specified in the USP XXV and conducted pursuant to the individual Official Monographs of USP XXV for the particular therapeutically active agent(s).

As used herein, “adduct formation” refers to the formation of a compound with a particular formulation of a composition by a solid phase reaction. The general term “adduct” for a compound, also called an addition compound, results from the direct combination of two or more different compounds. For example, in the present invention, lactose adduct formation (or other reducing sugars) may occur with formulations containing lactose (or other reducing sugars). Such adduct formation detracts from the efficacy of the product and increases the risks of other side effects.

A “therapeutically effective amount” means the amount of a compound that, when administered to a mammal for treating a state, disorder or condition is sufficient to effect a treatment (as defined below). The “therapeutically effective amount” will vary depending on the compound, the disease and its severity and the age, weight, physical condition and responsiveness of the mammal to be treated. According to the instant invention, in one embodiment, a therapeutically effective amount of memantine is an amount effective to treat CNS disorders, including Alzheimer's disease or Parkinson's disease. In another embodiment, a therapeutically effective amount is an amount effective to treat neuropathic pain, or other painful conditions such as visceral hypersensitivity. Other uses include, but are not limited to, the treatment of dementia, depression, and neuropathic pain. The effective amount of the drug for pharmacological action, and therefore the capsule strength, depends on the disease itself, e.g., in Alzheimer's disease, the patient is initially given a 5 mg dose and the dosage is progressively increased to 10 mg twice a day. Additional doses evaluated in clinical trials include 40 mg/day. In the present invention, e.g., in Alzheimer's disease treatment with the modified solid dosage form, the patient may be initially given 2.5 and increase to 80 mg, more preferably initially given 7 mg to 33 mg given once a day. Additionally, in the IR dosage form is given in about 4 to 5 increments. The modified release may be given in 3 to 4 increments due to its better tolerability.

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

As used herein, the term “treat”, in all its verb forms, is used herein to mean to relieve or alleviate at least one symptom of a disorder in a subject, the disorder including for example, pain, Alzheimer's disease, vascular dementia, or Parkinson's disease. The term “treat” may mean to relieve or alleviate the intensity and/or duration of a manifestation of a disorder experienced by a subject in response to a given stimulus (e.g., pressure, tissue injury, cold temperature, etc.). For example, in relation to dementia, the term “treat” may mean to relieve or alleviate cognitive impairment (such as impairment of memory and/or orientation) or impairment of global functioning (activities of daily living, ADL) and/or slow down or reverse the progressive deterioration in ADL or cognition. Within the meaning of the present invention, the term “treat” also denote to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject. Within the meaning of the present invention, the dementia is associated with a CNS disorder, including without limitation neurodegenerative diseases such as Alzheimer's disease (AD), Down's Syndrome and cerebrovascular dementia (VaD). The term “treatment” means the act of “treating” as defined above.

The term “dose proportional” as used herein refers to the relationship between the dose of a drug and its bioavailability. For example, dose proportionality exists if twice as much of the same composition will deliver twice the drug and provide the same bioavailability (e.g., AUC) as one dose of the dosage form. The dose proportionality of the present invention applies to a wide range of doses as discussed in detail herein.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value. For example, when referring to a period of time, e.g., hours, the present values (±20%) are more applicable. Thus, 6 hours can be, e.g., 4.8 hours, 5.5 hours, 6.5 hours, 7.2 hours, as well as the usual 6 hours.

The term “entry into a use environment” means contact of a formulation of the invention with the gastric or enteric fluids of the patient to whom it is administered, or with a fluid intended to simulate gastric fluid. As used herein, “use environment” refers to the stomach or other portion of the gastrointestinal tract intended as the site of major absorption locus for the drug.

The term “similarity factor” or f2 factor as used herein refers to one way of comparing dissolution profiles of two different products. (Multisource Pharmaceutical Products: Guidelines on Registration Requirements to establish Interchangeability, Quality Assurance and Safety: Medicines, Essential Drugs and Medicines Policy, World Health Organization, 1211 Geneva 27, Switzerland) This model independent mathematical approach compares the dissolution profile of the two products: test and reference (or two strengths, or pre- and post-approved products from the same manufacturer). Tests are recommended to be performed under the same test conditions. The dissolution time points for both the profiles should be the same, for example for immediate release products e.g. 10, 15, 30, 45, 60 minutes and for extended release products, e.g., 1, 2, 3, 5 and 8 hours. Only one time point should be considered after 85% dissolution of the reference product. An f₂ value of 50 or greater (50-100) ensures sameness or equivalence of the two curves, and thus the performance of the two products. The similarity factor f₂ should be computed using the equation:

f₂=50 log {[1+(1/n)ₜ₌₁ⁿ(Rₜ−Tₜ)²]^{-0.5}100}

where Rₜ and Tₜ are the cumulative percentage of the drug dissolved at each of the selected n time points of the comparator (reference) and (test) product respectively. For products which are very rapidly dissolving, i.e. more than 85% dissolution in 15 minutes or less, a profile comparison is not necessary. For extended release beaded capsules, where the strength differs only in the number of beads containing active moiety, dissolution profile comparison (f₂≧50) under one recommended test condition is sufficient for biowaivers. Whereas for extended release tablets, when the drug product is in the same dosage form but in a different strength, and is proportionally similar in its active and inactive ingredients and has the same drug release mechanism, a lower strength can be granted a biowaiver if it exhibits similar dissolution profiles, f₂≧50, in three diverse pH buffers (between pH 1.2 and 7.5) by the recommended test method.

The term “dissolution stability” as used herein refers to the similarity of dissolution profiles (similarity factor greater than 50, in comparison to initial) obtained at different periods of storage at varying temperature and humidity conditions.

The term “substantially the same dissolution stability” means similarity factor f2 of greater than 50 as compared to a reference dissolution profile.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Example 1

### Preparation of Memantine HCl Loaded Bead Forms (not MR)

The present example describes the general process of developing immediate release memantine hydrochloride loaded beads using povidone as a binder.

### 1. Preparation of Memantine HCl Suspension (Binder—Povidone)

Povidone USP is mixed with water, using a stirrer until it is fully dissolved. Memantine HCl is added to the container with the povidone solution and mixed for at least 15 minutes. Talc USP is added and mixing is continued for at least half an hour.

### 2. Coating of Memantine HCl Suspension Containing Povidone

Coat pre-warmed sugar spheres USP with a layer of the memantine HCl suspension using a fluidized bed coater such as GPCG3 (Glatt Fluid Air, Ramsey, N.J.). The coating is done at the following process parameters (for batch size=1.0 to 3.0 Kg):

Product temperature=43 to 51° C.

Air velocity=5 to 9 m/s

Spray rate=9 to 42 gm/min

Atomization pressure=1.5 to 2.0 bar

The coated beads are dried for 5 minutes in the fluidized bed. The beads are then discharged and stored in appropriate containers. The beads are coated with a drug layering suspension. The amount solids, i.e., weight gain on the core is dependent on the solids in formula.

### Example 2

### Preparation of Memantine HCl Loaded Bead Forms

The present example describes the general process of developing immediate release memantine hydrochloride loaded beads using an HPMC binder.

### 1. Preparation of Memantine HCl Suspension (Binder—HPMC (Opadry®, Colorcon, Pa.))

Hydroxypropyl methylcellulose (Opadry®) is mixed with water, using a stirrer, until it is fully dissolved to generate an Opadry® solution. Memantine HCl is added to the container with the Opadry® solution and mixed for at least 15 minutes. Talc USP is added and mixing is continued for at least half an hour.

### 2. Preparation of Seal-Coating Solution and Over-Coating Solution

The hydroxypropyl methylcellulose (Opadry®) is mixed with water, using a stirrer, until it is fully dissolved to obtain a 7% w/w solution.

### 3. Coating of Memantine HCl Suspension Containing Opadry

Sugar spheres, USP are coated with a layer of memantine HCl Suspension using a fluidized bed coater such as GPCG3 (Glatt Fluid Air, Ramsey, N.J.). This is done at the following process parameters (for batch size=1.0 to 3.0 Kg).

Product temperature=43 to 51° C.

Air velocity=5 to 9 m/s

Spray rate=9 to 42 gm/min

Atomization pressure=1.5 to 2.0 bar

Dry the coated beads at an inlet temperature of 45 to 50° C. in the fluid bed for 5-30 minutes. The beads are coated with a drug layering suspension. The amount of the solids, i.e. weight gain on the core is dependent on the solids in formula.

### Example 3

### Preparation of Memantine HCl Modified Release Bead Dosage Forms

The present example describes the general process of developing memantine hydrochloride modified release beads using an aqueous ethylcellulose dispersion.

1. Drug loaded beads are prepared according to Example 1 or 2.
2. Preparation of ethylcellulose dispersion (Surelease®, Colorcon, Pa.)

Mix Surelease® with water, using a stirrer for at least 15 minutes to obtain 15% w/w dispersion.

3. Coating with Surelease® polymer

The drug loaded beads are coated with ethylcellulose dispersion (Surelease®) using a fluidized bed coater, such as GPCG3 manufactured by Glatt fluid Air (Ramsey, N.J.). This is done at the following process parameters (for batch size=1.0 to 3.0 Kg):

Product temperature=38 to 45° C.

Air velocity=5 to 9 m/s

Spray rate=15 to 22 gm/min

Atomization pressure=1.0 to 2.0 bar

The target weight gain=3% w/w

The coated beads are dried at an inlet temperature of 45° to 50° C. in the fluid bed for 5 minutes.

### Example 4

### Preparation of Memantine HCl Modified Release Bead Dosage Forms

The present example describes the process of developing memantine hydrochloride modified release beads using an Eudragit® (Rhom Pharma, NJ) dispersion.

1. Drug loaded beads are prepared according to Example 1 or 2.

### 2. Preparation of Eudragit® RS/RL Dispersion

The Eudragit® RS 30D and RL 30D, which are 30% w/w aqueous dispersions, are weighed and combined at a ration of 95 to 5, in a suitable mixing tank and stirred for a period of 15 minutes using a mechanical stirrer. Triethyl citrate (TEC) is added to the Eudragit® mixture and mixed for 15 minutes to obtain a homogeneous dispersion. Talc, USP is weighed and transferred slowly to purified water in another suitable mixing tank and stirred for at least 30 minutes to obtain a homogeneous dispersion. The talc dispersion is added to the Eudragit®/TEC mixture and stirred for at least 30 minutes to obtain a homogeneous dispersion. The dispersion is then screened by passing through a #60 (250 μm) sieve.

3. Polymer coating using Eudragit® RS/RL Dispersion:

The drug loaded beads are coated with Eudragit® RS/RL dispersion using a fluidized bed coater such as GPCG3 manufactured by Glatt Air Techniques (Ramsey, N.J.). The coating is done at the following process parameters (for batch size=1.0 to 3.0 Kg):

Product temperature=22 to 27° C.

Air velocity=5 to 9 m/s

Spray rate=15 to 22 gm/min

Atomization pressure=1.0 to 2.0 bar

The target weight gain=6% w/w

The polymer coated beads are dried at an inlet temperature of 22 to 30° C. in the fluid bed for 30 minutes.

### Example 5

### Preparation of Memantine HCl Modified Release Bead Dosage Forms

The present example describes the process of developing memantine hydrochloride modified release beads using a methacrylic acid copolymer dispersion.

1. Drug loaded beads were prepared according to Example 1 or 2.
2. Preparation of Methacrylic Acid Copolymer Type C dispersion (Acryl-Eze®)

Acryl-Eze® was mixed with water, using a stirrer for at least 30 minutes.

### 3. Polymer Coating Using Methacrylic Acid Copolymer Type C Dispersion (Acryl-Eze®)

The drug loaded beads are coated with Methacrylic Acid Copolymer Type C dispersion (Acryl-Eze®) using a fluidized bed coater such as GPCG3 manufactured by Glatt Fluid Air (Ramsey, N.J.). The coating is done at the following process parameters (for batch size=1.0 to 3.0 Kg):

Product temperature=26 to 34° C.

Air velocity=5 to 9 m/s

Spray rate=15 to 22 gm/min

Atomization pressure=1.0 to 2.0 bar

The target weight gain=30% w/w

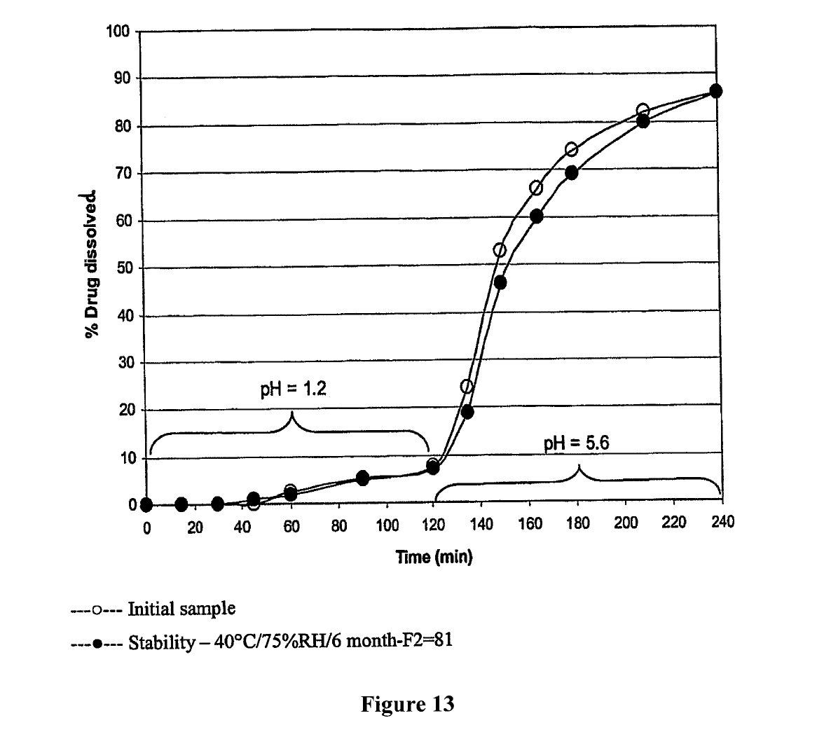

The coated beads are dried at an inlet temperature of 45° to 50° C. in the fluid bed for 5-30 minutes. Dissolution rates are shown in FIG. 13.

### Example 6

### Seal Coating and Over-Coating of Memantine HCl Modified Release Bead Dosage Forms

The present example describes the process of seal coating and over-coating memantine hydrochloride modified release beads.

1. Drug loaded beads were prepared according to one or more of Examples 1 through 6.
2. Seal coating and Over-coating

The drug loaded beads can be further seal coated with a layer of hydroxypropyl methylcellulose using the following process parameters (for batch size=1.0 to 3.0 Kg).

Product temperature=43 to 51° C.

Air velocity=5 to 9 m/s

Spray rate=9 to 16 gm/min

Atomization pressure=1.0 to 2.0 bar

Similarly, the Polymer coated beads can be further over coated with hydroxypropyl methylcellulose (Opadry®) to obtain a weight gain of 2% w/w. The coated beads are dried at an inlet temperature of 45° to 50° C. in the fluid bed for 5-30 minutes.

### Example 7

### Formulation of Memantine HCl IR Beads—with Povidone USP

This example shows the formulations of memantine HCl immediate release beads with Povidine USP as a binder at 100 mg/g and 171 mg/g.

TABLE 9

| | | Amount | Amount |
|---|---|---|---|
| Ingredients | | (mg/g) | (mg/g) |
| Active Ingre- | Memantine HCl | 100 | 171 |
| dient (AI) | | | |
| Binder to AI | Povidone, USP | 10 | 17 |
| Glidant | Talc, USP | 10 | 17 |
| Core | Sugar Spheres, USP | 880 | 795 |
| Inert | Purified Water, USP* | | NA |
| | Total (Drug Loaded Beads) | 1000 | 1000 |

*Purified Water is removed during the process

The process of preparation of these beads involves the following steps:

1. Preparation of memantine HCl Suspension (Binder—Povidone)
2. Coating of memantine HCl Suspension containing Povidone

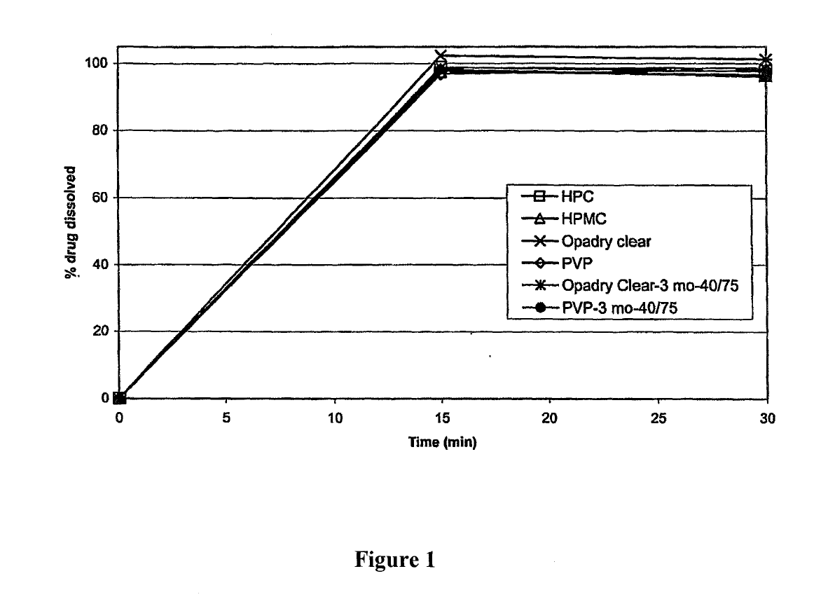

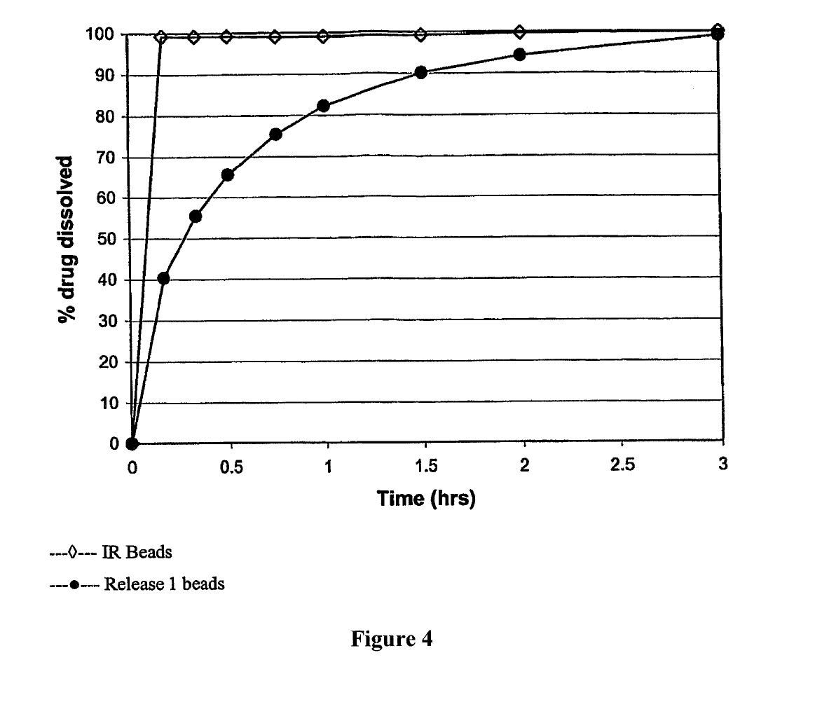

Dissolution data for these beads are provided in FIG. 1. In FIG. 4, a comparison of dissolution of IR beads with Release 1 beads shows a similarity factor F2 of only about 26, which means that the release profiles are substantially different showing that modified release is achieved with the 3% coating level.

### Example 8

### Formulation of Memantine HCl IR Beads—with Opadry Clear

This example shows the formulation of memantine HCl immediate release beads with Opadry® Clear as the binder (157 mg/g).

TABLE 10

| | | Amount |
|---|---|---|
| Ingredients | | (mg/g) |
| Active Ingre- | Memantine HCl | 157 |
| dient (AI) | | |
| Binder to AI | Hydroxypropyl | 157 |
| | Methylcellulose (Opadry ®) | |
| Glidant | Talc, USP | 16 |
| Core | Sugar Spheres, USP | 651 |
| Coating | Hydroxypropyl | 19 |
| | Methylcellulose (Opadry ®) | |
| Inert | Purified Water, USP* | NA |
| | Total (Seal Coated Beads) | 1000 |

*Purified Water is removed during the process

The process of preparation of these beads involves the following steps:

1. Preparation of memantine HCl Suspension—Binder—HPMC (Opadry®);

### 2. Preparation of Seal Coating Solution; and

3. Coating of memantine HCl Suspension containing Opadry and Seal Coating.

Dissolution data for these beads are provided in FIG. 1. In FIG. 1, the high F2 values (>50) for comparison of initial dissolution with that of stored samples indicate good dissolution stability.

### Example 9

### Formulation of Memantine HCl Release Beads (Release 1 and 3)

This example shows the formulation of memantine HCl immediate release beads with Surelease® 3% with PVP, and Surelease® 6% with PVP. The process of preparation of these beads involves the following steps:

1. Preparation of memantine HCl Suspension (Binder—Povidone);
2. Preparation of Ethylcellulose dispersion (Surelease®);
3. Preparation of over-coating solution;
4. Coating of memantine HCl Suspension containing Povidone;
5. Coating with Surelease® polymer; and

### 6. Over-coating.

TABLE 11
Release 1 (R1) beads, Surelease ® 3% PVP

| | Amount | |
|---|---|---|
| Ingredients | (mg/g) | Process Step |
| Memantine HCl (Active Ingredient) | 163 | Drug Loading |
| Povidone, USP (Binder to AI) | 16 | |
| Talc, USP (Glidant) | 16 | |
| Sugar Spheres, USP (Core) | 756 | |
| Ethylcellulose (Surelease ®) | 115 | Release |
| | | Modifying |
| | | Polymer |
| | | Coating |
| Hydroxypropyl Methylcellulose (Opadry ®) | 20 | Over-coating |
| Purified Water, USP* | NA | |
| Total | 1000 | |

*Purified Water is removed during the process
¹ Contains 25% w/w solids.

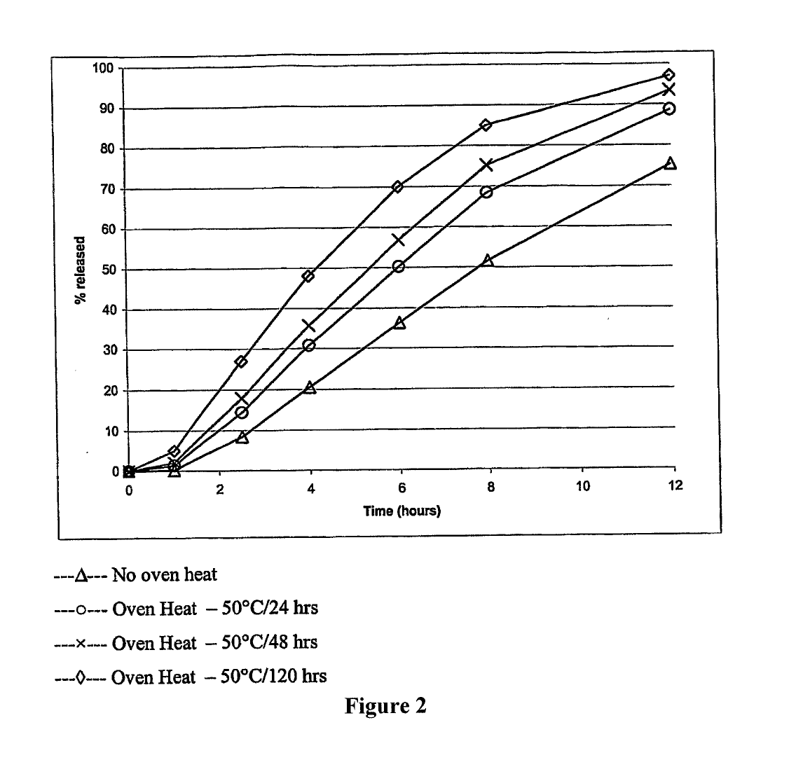

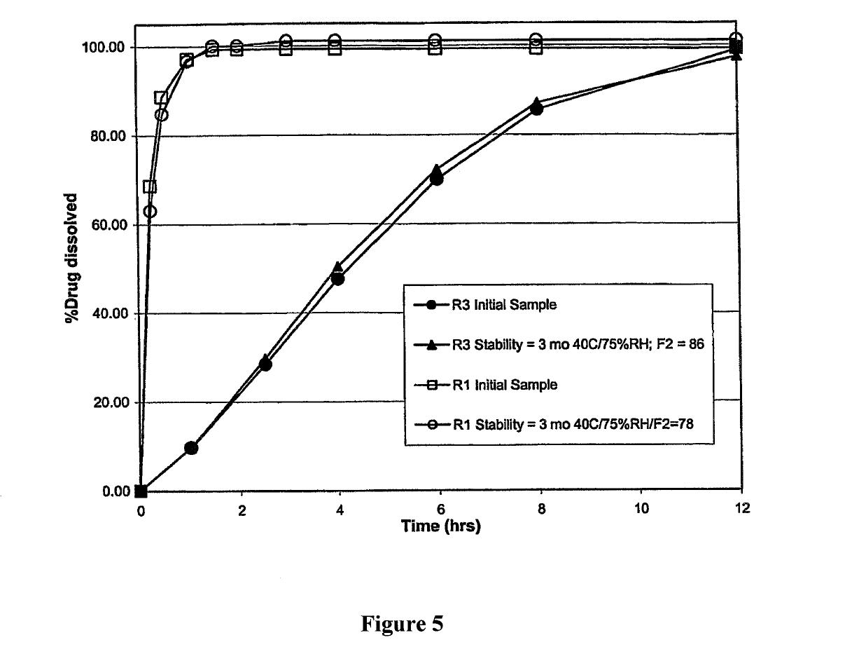

The modified release rate required is achieved and does not change substantially after heating the beads at 50° C. (See FIG. 2). Comparison of dissolution of IR beads with Release 1 beads is provided in FIG. 4. Dissolution stability data is provided in FIG. 5. High F2 values (>50) obtained in comparison of initial dissolution rate with that of the stored samples indicate excellent dissolution stability.

TABLE 12
Memantine HCl Release 3 beads, Surelease ® 6%/PVP

| | Amount | |
|---|---|---|
| Ingredients | (mg/g) | Process Step |
| Memantine HCl (Active Ingredient) | 159 | Drug Loading |
| Povidone, USP (Binder to AI) | 16 | |
| Talc, USP (Glidant) | 16 | |
| Sugar Spheres, USP (Core) | 734 | |
| Ethylcellulose (Surelease ®) | 222 | Release |
| | | Modifying |
| | | Polymer |
| | | coating |
| Hydroxypropyl Methylcellulose (Opadry ®) | 20 | Over-coating |
| Purified Water, USP*⁴ | NA | |
| Total | 1000 | |

*Purified Water is removed during the process
¹Contains 25% w/w solids.

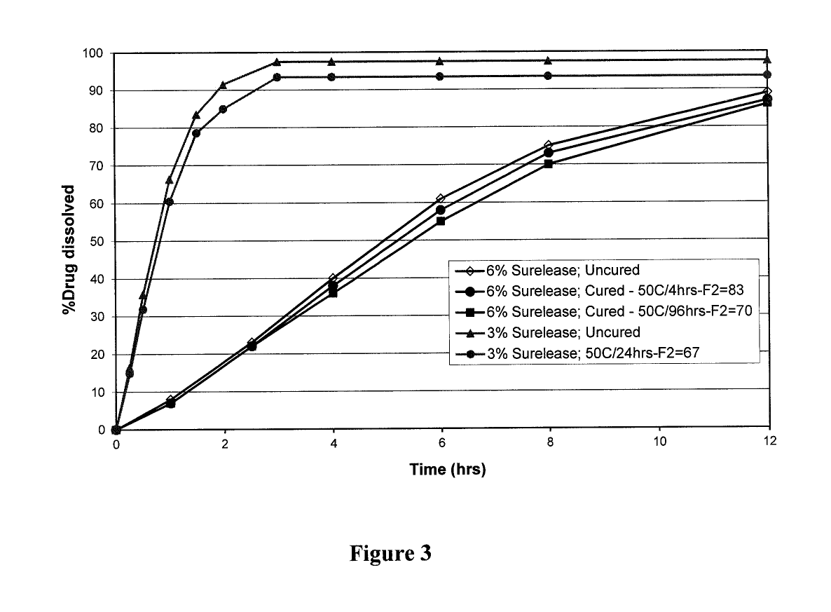

Dissolution data for these beads is provided in FIG. 5. The effect of oven heating on these beads is also illustrated in FIG. 5. The data show no substantial change in dissolution rate after heating the beads for short duration at 40° C. and 50° C. Dissolution rate stability is shown in FIG. 3. Similarly, modified release bead with different levels of Surelease, weight gain can be prepared with IR beads 171 mg/g or 100 mg/g.

### Example 10

### Formulation of Memantine HCl Modified Release Beads (Release 4, 5 and 6)

This example shows the formulation of memantine HCl immediate release beads with Eudragit/HPMC at 6% w/w, 10% w/w, and 20% w/w Eudgragit®. The process of preparation of Eudragit/HPMC beads involves the following steps:

1. Preparation of memantine HCl Suspension (Binder—HPMC (Opadry®))

### 2. Preparation of Seal Coating Solution

### 3. Preparation of Eudragit® RS/RL Dispersion

### 4. Preparation of Over—Coating Solution

5. Coating of memantine HCl Suspension containing Opadry®

### 6. Seal Coating

7. Polymer coating with Eudragit® RS/RL Dispersion

### 8. Over-coating

TABLE 13
Release 4 beads Eudragit ® 6% w/w/HPMC

| | Amount | |
|---|---|---|
| Ingredients | (mg/g) | Process Step |
| Memantine HCl (Active Ingredient) | 144 | Drug loading |
| Hydroxypropyl Methylcellulose (Opadry ®) | 144 | |
| (Binder to AI) | | |
| Talc, USP (Glidant) | 14 | |
| Sugar Spheres, USP (Core) | 598 | |
| Hydroxypropyl Methylcellulose (Opadry ®) | 18 | Seal-coating |
| Ammonio Methacrylate Copolymer NF | 87 | Modified |
| (Eudragit ®)* | | |
| Ammonio Methacrylate Copolymer NF Type A, | 5 | Release |
| (Eudragit ®)* | | Polymer |
| Triethyl Citrate, NF | 7 | coating |
| Talc, USP | 28 | |
| Hydroxypropyl Methylcellulose (Opadry ®) | 19 | Over-coating |
| Purified Water, USP¹ | NA | |
| Total (Seal Coated Beads) | 1000 | |

*Contains 30% w/w solids
¹Purified Water is removed during the process

TABLE 14
Memantine HCl Release 5 beads, Eudragit ® 10% w/w/HPMC

| | Amount | |
|---|---|---|
| Ingredients | (mg/g) | Process Step |
| Memantine HCl (Active Ingredient) | 136 | Drug loading |
| Hydroxypropyl Methylcellulose (Opadry ®) | 136 | |
| (Binder to AI) | | |
| Talc, USP (Glidant) | 14 | |
| Sugar Spheres, USP (Core) | 568 | |
| Hydroxypropyl Methylcellulose (Opadry ®) | 17 | Seal-coating |
| Ammonio Methacrylate Copolymer NF* | 152 | Modified |
| (Eudragit ®) | | |
| Ammonio Methacrylate Copolymer NF Type A, | 12 | Release |
| (Eudragit ®)* | | |
| Triethyl Citrate, NF | 12 | Polymer |
| Talc, USP | 48 | coating |
| Hydroxypropyl Methylcellulose | 19 | Over-coating |
| (Opadry ®) | | |
| Purified Water, USP¹ | NA | |
| Total (Seal Coated Beads) | 1000 | |

*Contains 30% w/w solids
¹Purified Water is removed during the process

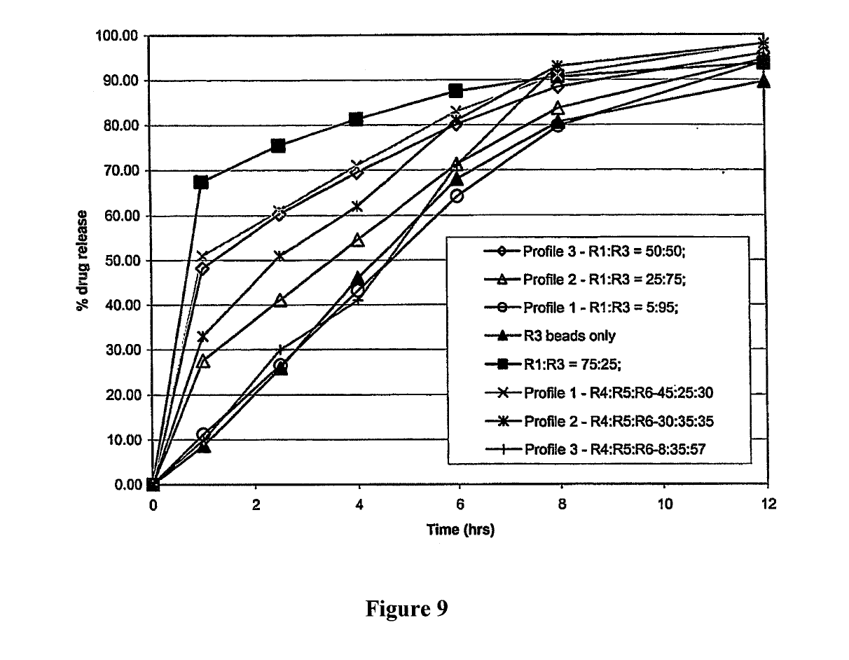

Dissolution stability data for these beads is provided in FIGS. 3 and 9.

TABLE 15
Memantine HCl Release 6 beads, Eudragit ® 20% w/w/HPMC

| | Amount | |
|---|---|---|
| Ingredients | (mg/g) | Process Step |
| Memantine HCl (Active Ingredient) | 123 | Drug loading |
| Hydroxypropyl Methylcellulose (Opadry ®) | 123 | |
| (Binder to AI) | | |
| Talc, USP (Glidant) | 12 | |
| Sugar Spheres, USP (Core) | 511 | |
| Hydroxypropyl Methylcellulose (Opadry ®) | 15 | Seal-coating |
| Ammonio Methacrylate Copolymer NF | 273 | Modified |
| (Eudragit ®)* | | |
| Ammonio Methacrylate Copolymer NF Type A, | 22 | Release |
| (Eudragit ®)* | | |
| Triethyl Citrate, NF | 22 | Polymer |
| Talc, USP | 86 | coating |
| Hydroxypropyl Methylcellulose (Opadry ®) | 19 | Over-coating |
| Purified Water, USP¹ | NA | |
| Total (Seal Coated Beads) | 1000 | |

*Contains 30% w/w solids
¹Purified Water is removed during the process

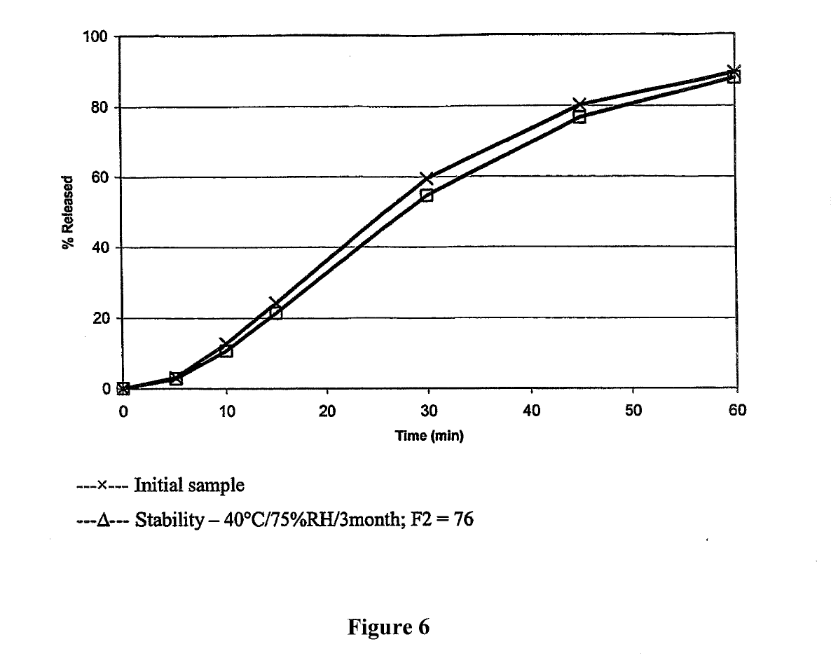

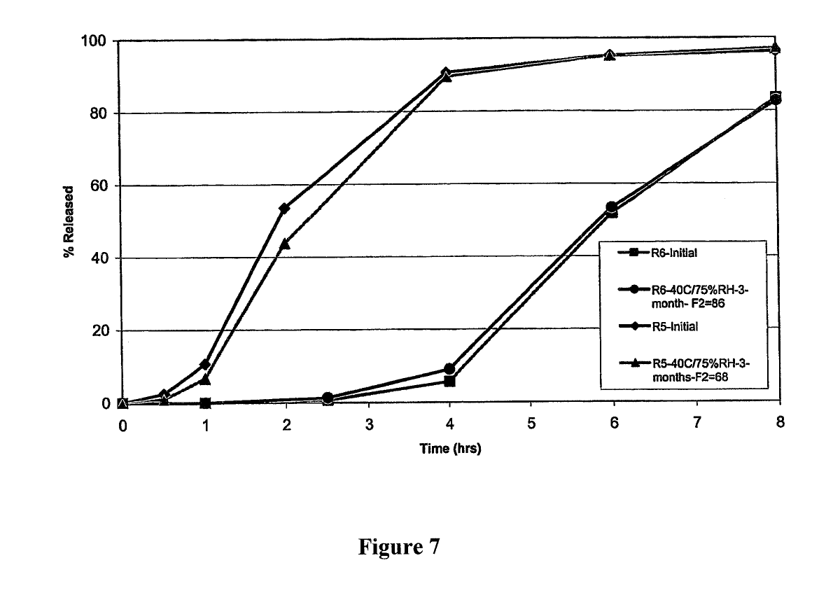

In this example, HPMC was used as a binder and Eudragit® as the release modifying polymer. Neither shows substantial difference in dissolution rate after heat for short periods of time at 50° C. Dissolution stability data is provided in FIGS. 6 and 7.

### Example 11

### Formulation of Memantine HCl Modified Release Beads

This example shows the formulation of memantine HCl modified release beads with Acryl-Eze® polymer.

TABLE 16

| | Amount | |
|---|---|---|
| Ingredients | (mg/g) | Process Step |
| Memantine HCl (Active Ingredient) | 91.9 | Drug loading |
| Sugar spheres, USP (Core) | 570.4 | |
| Hydroxypropyl Methylcellulose (Opadry ®) | 91.9 | |
| (Binder to AI) | | |
| Hydroxypropyl Methylcellulose (Opadry ®) | 15.0 | Seal-coating |
| Methacrylic Acid Copolymer Type C, NF | 230.8 | Modified |
| (Acryl-Eze ®) | | Release |
| | | Polymer |
| | | coating |
| Purified Water, USP¹ | NA¹ | |
| Total (Seal Coated Beads) | 1000 | |

¹Purified Water is removed during the process

The process of preparation of these beads involves the following steps:

1. Preparation of memantine HCl Suspension (Binder—HPMC (Opadry®))

### 2. Preparation of Seal Coating Solution

### 3. Preparation of Acryl-Eze® Dispersion

4. Coating of memantine HCl Suspension containing Opadry®

### 5. Seal Coating

6. Polymer coating with Acryl-Eze® Dispersion

Dissolution rates are shown in FIG. 13.

### Example 12

### Preparation of Unitary Modified Release Capsules

This example demonstrates the preparation of dose proportional unitary capsules based on beads prepared from Example 9, specifically release 3. The capsules presented below include 2.5 mg, 7 mg, 14 mg, 21 mg, 28 mg, 40 mg, 80 mg and 100 mg formulations.

TABLE 17

| | | Capsule |
|---|---|---|
| Strength (mg) | Fill weight | size required |
| 2.5 | 15.8 | 5 |
| 6 | 38.7 | 5 |
| 7 | 44.1 | 5 |
| 14 | 88.3 | 5 |
| 21 | 132.4 | 4 |
| 28 | 176.6 | 3 |
| 40 | 252.3 | 1 |
| 60 | 387.4 | 0 |
| 80 | 504.6 | 0 |
| 100 | 630.7 | 00 |

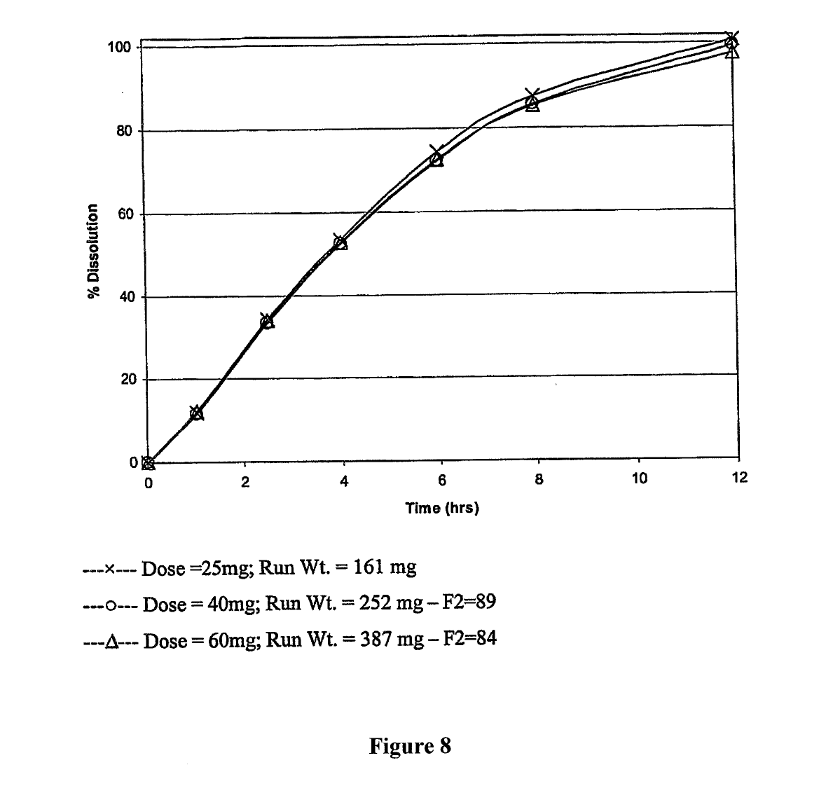

Prepare the encapsulation machine (MG-2 Futura, N.J.) for appropriate size capsules. Fill the capsules with memantine HCl MR Beads, Release 3. The fill weight is for all the strengths and capsule sizes are provided in Table 9. Inspect the weight of all individual capsules using Weigh Inspection Equipment. In addition, MR bead prepared with different IR beads and coating levels may be prepared. Dissolution data for 25, 40 and 60 mg strengths is provided in FIG. 8.

### Example 13

### Preparation of Capsules with Plurality of Modified Release Beads (40 mg)

This example demonstrates the preparation of capsules with Release 1 and Release 3 beads in various ratios.

Prepare the encapsulation machine for size capsules. Fill the capsules with memantine HCl MR Beads, e.g. Release 1 and Release 3. The fill weight is shown below for different dose ratios. Inspect the weight of all individual capsules using Weigh Inspection Equipment.

TABLE 18

| | Amount | Amount | Amount | Amount | |
|---|---|---|---|---|---|
| | (mg/ | (mg/ | (mg/ | (mg/ | Amount |
| Ingredient | capsule) | capsule) | capsule) | capsule) | (mg/capsule) |
| Memantine HCl | 239.7 | 183.9 | 122.6 | 61.3 | 12.3 |
| Release 1 beads | | | | | |
| Memantine HCl | 12.3 | 63.1 | 126.1 | 189.2 | 239.7 |
| Release 3 beads | | | | | |
| Hard Gelatin | 118 | 118 | 118 | 118 | 118 |
| capsule size 00 | | | | | |
| Total | 370.0 | 365.0 | 366.7 | 368.5 | 370.0 |
| Dose ratio | 5:95 | 30:10 | 20:20 | 10:30 | 2:38 |
| Release 1:Release 3 | | | | | |

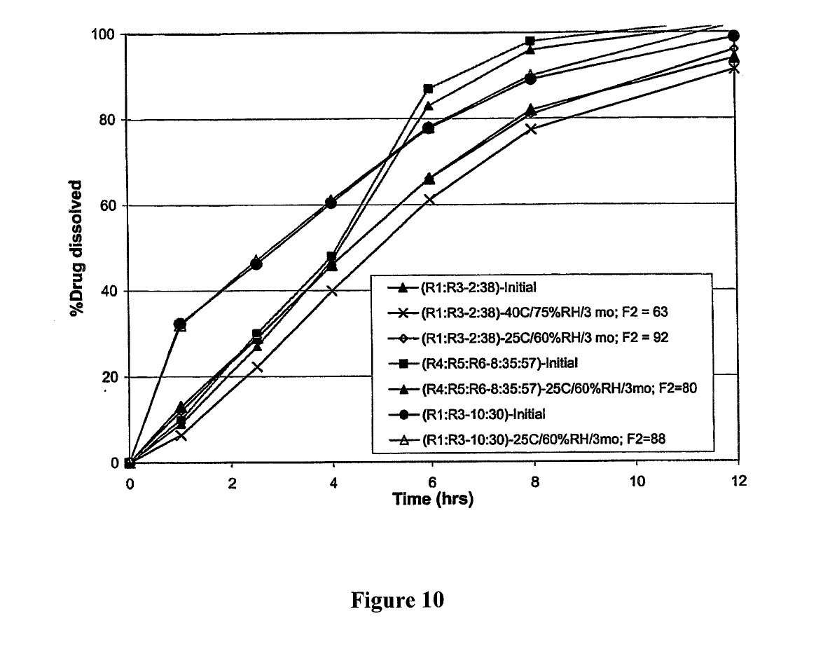

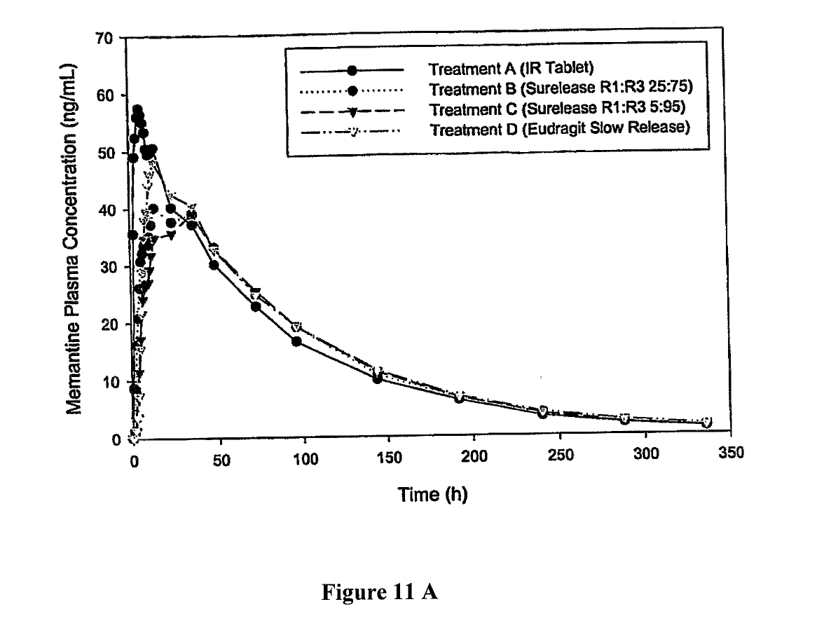

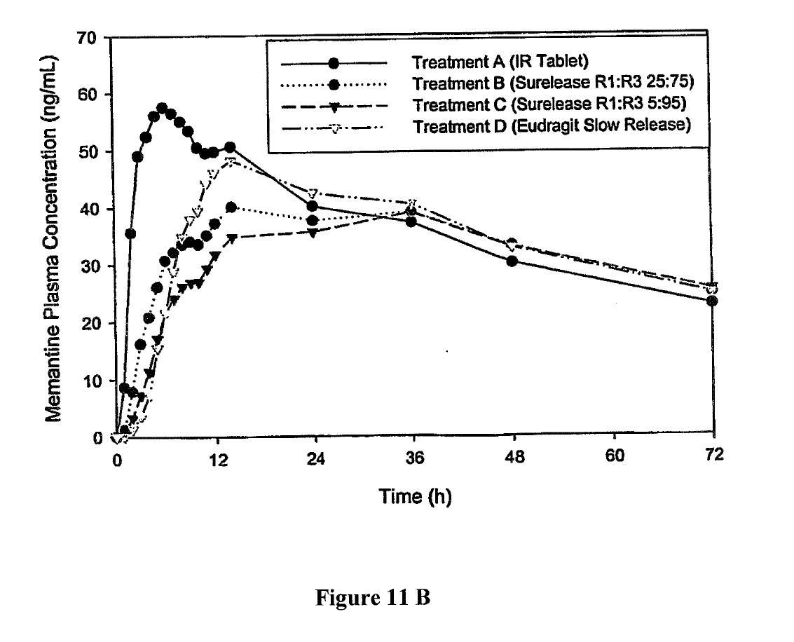

Dissolution data for these capsules are provided in FIG. 10. As shown in FIG. 10, high F2 values (>50) obtained on comparison of the initial dissolution rate with that of the stored sample indicate excellent dissolution stability. The blood plasma concentration values of memantine are provided in FIG. 11.

Tables 19-21 show the individual capsules formulations of memantine, Surelease® coated beads and the applicable ranges of bead weights that may be employed.

TABLE 19
Profile 1 (slow) Capsules with plurality of
beads 40 mg Example (R1:R3 = 5:95)

| | | | |
|---|---|---|---|
| | Amount | Range | Preferred |
| Ingredient | (mg/capsule) | (mg/g) | range (mg/g) |

| | | | |
|---|---|---|---|
| Memantine HCl Release 1 beads | 239.7 | 215-265 | 230-250 |
| Memantine HCl Release 3 beads | 12.3 | 11-14 | 12-13 |
| Hard Gelatin capsule | 118 | | |
| Total | 370.0 | | |

TABLE 20
Profile 2 (medium) Capsules with plurality of beads 40 mg
Example (R1:R3 = 25:75)

| | Amount | Range | Preferred |
|---|---|---|---|
| Ingredient | (mg/capsule) | (mg/g) | range (mg/g) |
| Memantine HCl Release 1 beads | 61.3 | 55-67 | 58-64 |
| Memantine HCl Release 3 beads | 189.2 | 170-210 | 180-200 |
| Hard Gelatin capsule size 00 | 118 | | |
| Total | 368.5 | | |

TABLE 21
Profile 3 (fast) Capsules with plurality of
beads 40 mg Example (R1:R3 = 50:50)

| | Amount | Range | Preferred |
|---|---|---|---|
| Ingredient | (mg/capsule) | (mg/g) | range (mg/g) |
| Memantine HCl Release 1 beads | 122.6 | 110-135 | 115-130 |
| Memantine HCl Release 3 beads | 126.1 | 115-140 | 20-130 |
| Hard Gelatin capsule | 118 | | |
| Total | 366.7 | | |

### Example 14

### Preparation of Capsules with Plurality of Beads (40 mg)

This example demonstrates the preparation of capsules with Release 4, Release 5 and Release 6 beads in various ratios.

Prepare the encapsulation machine for size capsules. Fill the capsules with memantine HCl MR Beads, e.g. Release 4, Release 5 and Release 6. The fill weight is shown below for different dose ratios. Inspect the weight of all individual capsules using Weigh Inspection Equipment.

TABLE 22

| | | | Amount |
|---|---|---|---|
| | Amount | Amount | (mg/ |
| Ingredient | (mg/capsule) | (mg/capsule) | capsule) |
| Memantine HCl Release 4 beads | 22.2 | 83.6 | 125.5 |
| Memantine HCl Release 5 beads | 101.4 | 101.4 | 72.4 |
| Memantine HCl Release 6 beads | 181.7 | 111.6 | 95.7 |
| Hard Gelatin capsule size 00 | 118 | 118 | 118 |
| Total | 423.3 | 414.6 | 411.6 |
| Dose Ratio | 3.2:14:22.8 | 12:14:14 | 18:10:12 |
| R4:R5:R6 | | | |

Dissolution data for these capsules are provided in FIG. 10. The blood plasma concentration values of memantine are provided in FIG. 11.

Tables 22-24 show the individual capsules formulations of memantine Eudragit coated beads and the applicable ranges of bead weights that may be employed.

TABLE 23
Profile 1 (slow) Capsules with plurality of beads 40 mg
(R4:R5:R6 = 3.2 mg:14 mg:22.8 mg)

| | | | Preferred |
|---|---|---|---|
| | Amount | Range | Range |
| Ingredient | (mg/capsule) | (mg/cap) | (mg/cap) |
| Memantine HCl Release 4 | 22.2 | 20.0-24.4 | 21.1-23.3 |
| beads | | | |
| Memantine HCl Release 5 | 101.4 | 91.3-111.5 | 96.3-106.5 |
| beads | | | |
| Memantine HCl Release 6 | 181.7 | 163.5-199.9 | 172.6-190.8 |
| beads | | | |
| Hard Gelatin capsule | 118 | | |
| Total | 423.3 | | |

TABLE 24
Profile 2 (medium) Capsules with plurality of beads 40 mg
(R4:R5:R6 = 12 mg:14 mg:14 mg)

| | | | Preferred |
|---|---|---|---|
| | Amount | Range | Range |
| Ingredient | (mg/capsule) | (mg/cap) | (mg/cap) |
| Memantine HCl Release 4 | 83.6 | 75.2-92.0 | 79.4-87.8 |
| beads | | | |
| Memantine HCl Release 5 | 101.4 | 91.3-111.5 | 96.3-106.5 |
| beads | | | |
| Memantine HCl Release 6 | 111.6 | 100.4-122.8 | 106.0-117.2 |
| beads | | | |
| Hard Gelatin capsule | 118 | | |
| Total | 414.6 | | |

TABLE 25
Profile 3 (fast) Capsules with plurality of beads 40 mg Example
(R4:R5:R6 = 18 mg:10 mg:12 mg)

| | | | Preferred |
|---|---|---|---|
| | Amount | Range | Range |
| Ingredient | (mg/capsule) | (mg/cap) | (mg/cap) |
| Memantine HCl Release 4 | 125.5 | 113.0-138.1 | 119.2-131.8 |
| beads | | | |
| Memantine HCl Release 5 | 72.4 | 65.2-79.6 | 68.8-76.0 |
| beads | | | |
| Memantine HCl Release 6 | 95.7 | 86.1-105.3 | 90.9-100.5 |
| beads | | | |
| Hard Gelatin capsule | 118 | | |
| Total | 411.6 | | |

### Example 15

### Pharmacokinetic Study of Memantine Formulations

The present example compares the bioavailability of three modified release bead memantine dosage forms as compared to immediate release memantine tablets. Current clinical uses of memantine as a marketed product and in clinical trials utilize a twice daily dosing regimen of immediate release tablets. The modified release bead formulation aimed for once daily dosing, to lower the Cₘₐₓ, and at the same time result in improved tolerability. The modified release formulation intended to provide exposure that would be sufficient for dosing once a day. The desired profile was set as a reduction of Cmax of at least 25% relative to IR tablet, without lowering the AUC by more than 20%.

### Subjects and Methods

A single center, open-label, randomized, four-way crossover study in 24 healthy young male and female subjects, naïve with respect to memantine, ages 18-45 (inclusive) was performed. 24 patients were enrolled, however 22 completed the study. Patients were screened within 14 days of the study start and included a medical history evaluation, complete physical examination (including blood pressure, pulse, temperature, height, weight and respiration rate), clinical laboratory evaluations ([consisting of hematology (including differential), chemistry and urinalysis), drugs of abuse screen (including alcohol and cotinine), HBsAg, anti-HCV screen, RPR/VDRL, Anti-HIV 1 and 2 tests, and a 12 lead ECG. Female subjects will have a β-HCG serum pregnancy test performed at screening. Abnormal (or positive) values in any of these tests were grounds for exclusion. The clinical laboratory tests included the following:
Hematology: Hemoglobin, hematocrit, RBC count, WBC count, WBC differential (percentages and absolute value) and platelet count.
Chemistry: Alkaline phosphatase, ALT, AST, total bilirubin, cholesterol, triglycerides, LDH, total protein, glucose, uric acid, BUN, creatinine, sodium, calcium, inorganic phosphorous and potassium.
Urinalysis: Specific gravity, pH, ketone bodies, protein, blood, glucose, bilirubin and microscopy (RBC/HPF, WBC/HPF, casts/LPF).

The drugs of abuse screen included benzoylecgonine (cocaine), methadone, barbiturates, amphetamines, benzodiazepine, cotinine, alcohol, cannabinoids, opiates and phencyclidine. Subjects were also tested for the use of tricyclic antidepressants. Subjects with a known hypersensitivity to memantine or other N-methyl-D-aspartate (NMDA) antagonists, hypertension, hypotension, heart abnormalities or disease, or a history of substance abuse were excluded. Concomitant medications were not permitted, nor the use of caffeine or other xanthine compounds. Subjects did not engage in strenuous activity at any time during the study.

The subjects received the following treatments, in a randomized order, each separated by a 21 day washout period:
Treatment A: Single dose of memantine 40 mg (two 20 mg immediate release tablets given at 0800);
Treatment B: Single dose of memantine 40 mg capsule (MR) Formulation I (Surelease® R1:R3 25:75) given at 0800 hours;
Treatment C: Single dose of memantine 40 mg capsule (MR) Formulation II (Surelease® R1:R3 5:95) given at 0800 hours; and
Treatment D: Single dose of memantine 40 mg capsule (MR) Formulation III (Eudragit® slow release) given at 0800 hours.

The study duration was 79 days (Day 1 through the last PK sample on day 78). 22 subjects completed the study.

Blood samples were collected by a qualified phlebotomist via venipuncture of the ante-cubital veins from either arm using purple top Vacutainer® tubes (containing tri-potassium EDTA as an anticoagulant). A 5 mL tube was used to collect the samples for the determination of memantine concentrations. Ninety-six (96) blood samples (5 mL each) per subject were collected. Approximately 510 mL of blood was collected per subject during the study (480 mL plus an additional 30 mL for pre-study and post-study clinical analysis). Blood plasma concentrations of memantine were measured at the following time intervals to determine the principal pharmacokinetic parameters: Days 1-5, 7, 9, 11, 13, 15, 22-26, 28, 30, 32, 34, 36, 43-47, 49, 51, 53, 55, 57, 64-68, 70, 72, 74, 76 and 78. On days 1, 22, 43, 64, sampling was done pre-dose, and at 1, 2, 3, 4, 5, 6, 7, 8, 9, 10, 11, 12, 14, 24, 36, 48, 72, 96, 144, 192, 240, 288 and 336 hours post dose. Subjects remained ambulatory or seated upright and awake for the first four hours following drug administration on Days 1, 22, 43 and 64.

A pre-chilled 5-mL Vacutainer® tube (containing tri-potassium EDTA as an anticoagulant) was used to collect blood samples for determination of memantine concentrations. Blood samples were centrifuged within thirty (30) minutes from the time of draw at no less than 2,500 g for 10 minutes at 4° C. and the plasma harvested. After centrifugation, the plasma samples were transferred into pre-chilled, coded polypropylene tubes. The samples were then flash frozen in an isopropyl alcohol/dry ice bath and stored at approximately −70° C.

Pharmacokinetic criteria were evaluated for rate and extent of bioavailability of memantine. Safety criteria was also evaluated to monitor clinical laboratory tests, adverse events, physical examinations, ECG, and vital signs. Vital signs were checked on the following days: days 1, 2, 22, 23, 43, 44, 64, 65, and 78. Blood pressure and pulse rate were measured in the sitting position (subjects must be sitting for at least 5 minutes), on the same arm throughout the study and before any corresponding blood sample was collected. In addition to the pre- and post-study measurement, vital signs were taken at the following times: on Days 1, 22, 43 and 64: pre-dose, 2, 4, 6, 8 and 24 hours after the 0800 hour dose administration.

### Statistical Methods

Pharmacokinetic parameters were compared by analysis of variance (ANOVA) using SAS® version 6.12 or later under the UNIX operating system. A general linear model with sequence, subject within sequence, treatment, and period as factors were used as the basis for the analysis. Statistical inference was based on log-transformed values for the Cₘₐₓ and AUC parameters and observed values for T_{1/2}.

The two-sided 90% confidence interval for the ratio of average AUC between each test formulation (MR capsules) and the reference formulation (IR tablet) was constructed.

Tₘₐₓ for test and reference were compared using the Wilcoxon signed-rank test using the non-parametric Wilcoxon signed rank test based on untransformed data.

Safety parameters (adverse experiences, vital signs, clinical laboratory evaluations, and ECG parameters) were summarized for all subjects. Adverse events and vital signs were also summarized by treatment. Incidence tables were prepared for adverse experiences categorized by severity and relationship to study drug. For other safety parameters, descriptive statistics were calculated. Subjects with potentially clinically significant post-baseline values of vital signs, laboratory parameters, and ECG parameters are noted.

Any AE occurring subsequent to the first dose of study medication, regardless of the relationship to study drug, was counted as a treatment emergent AE (TEAE), either if it was not present at baseline or if it was present at baseline but increased in severity during the treatment period. An Adverse Event or Adverse Experience (AE) was defined as any untoward medical occurrence in a subject or clinical investigation subject administered a pharmaceutical product. It was not necessary that the AE have a causal relationship to treatment with the product.

An AE therefore was any unfavorable and unintended sign (for example, a clinically significant abnormal laboratory finding) symptom, or disease temporally associated with the use of study medication, whether or not considered related to study medication. AEs included: Changes in the general condition of the subject; Subjective symptoms offered by or elicited from the subject; Objective signs observed by the Investigator or study personnel; or all concurrent diseases that occur after the start of the trial, including any change in severity or frequency of pre-existing disease; all clinically relevant laboratory abnormalities or physical findings that occur during the trial.

Causal relationship of each AE was classified according to the following criteria:
Related Reasonable temporal relation to study medication administration, AND cannot be reasonably explained by other factors (such as the subject's clinical state, concomitant therapy, and/or other interventions)
OR application/injection site reaction.
Possibly Relationship to study medication cannot be ruled out.
Related
Not Related Data are available to identify a clear alternative cause for the reaction (e.g., positive test for viral antigen in a case of suspected drug-induced hepatitis, hemorrhage due to mechanical injury).

Severity was assessed according to the following scale:
Mild The AE was an annoyance to the subject, but did not further hinder baseline functioning; the AE may have been intermittent or continuous.
Moderate The AE caused the subject to experience some discomfort or some interference with normal activities, but was not hazardous to health; prescription drug therapy may have been employed to treat the AE.
Severe The AE caused the subject to experience severe discomfort or severely limited or prevented normal activities and represented a definite hazard to health; prescription drug therapy and/or hospitalization may have been employed to treat the AE.

### Pharmacokinetic Parameters

The following pharmacokinetic parameters included area under the plasma concentration-time curve (AUC₀₋ₜ and AUC_{0-∞}), maximum plasma concentration (Cₘₐₓ), time of maximum plasma concentration (Tₘₐₓ) and terminal elimination half-life (T_{1/2}). The maximum plasma concentration of memantine was determined observationally as the peak concentration for each subject. The time of maximum concentration, Tₘₐₓ, was determined as the time corresponding to Cₘₐₓ. Area under the plasma concentration-time curve up to the time corresponding to the last measurable concentration (AUC₀₋ₜ) was calculated by numerical integration using the linear trapezoidal rule as follows:

$${AUC}_{0 - t} = {\sum }_{i = 2}^{n} 0.5 * ( {C}_{i} + {C}_{i - 1} ) * ( {t}_{i} - {t}_{i - 1} ) \qquad Eq . 1$$

where Cᵢ is the plasma memantine concentrations at the corresponding sampling time point tᵢ and n is the number of time points up to and including the last quantifiable concentration.

Estimates of the terminal half-life (T_{1/2}) were calculated using the following equation:

$${T}_{1 / 2} = \frac{0.693}{{λ}_{z}} \qquad Eq . 2$$

where λ_{z} is the terminal elimination rate constant.
The area under the plasma concentration-time curve from time zero to infinity was calculated according to the following equation:

$${AUC}_{0 - ∞} = {AUC}_{0 - t} + \frac{{C}_{last}}{{λ}_{z}} \qquad Eq . 3$$

where Cₗₐₛₜ is the last measurable concentration.

### Results

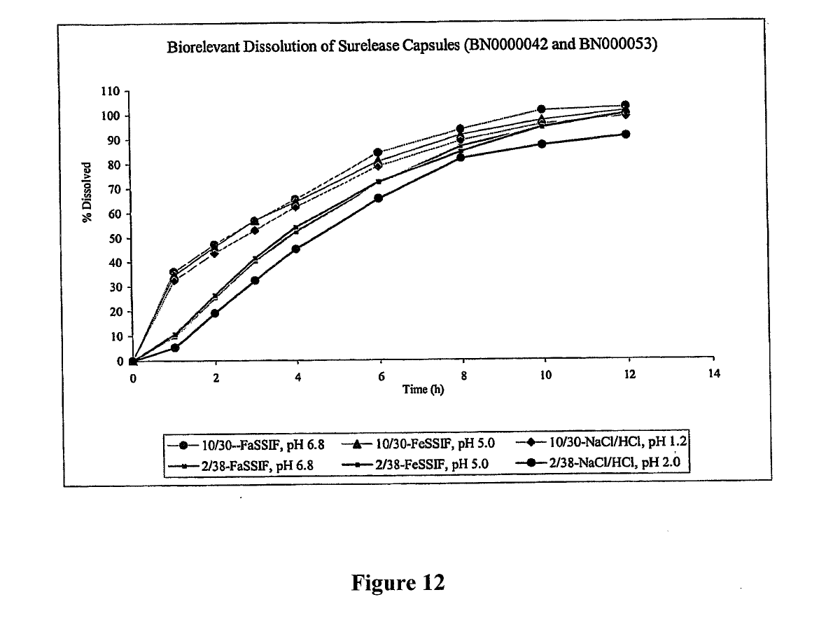

Serial plasma samples were collected after dose administration for analysis of memantine concentrations. The mean plasma concentration-time profiles following Treatment A, B, C, and D are presented in FIG. 11A. A truncated concentration-time profile is shown in FIG. 11B. FIG. 12 depicts the dissolution profiles for the 40 mg capsule containing a plurality of beads in different biorelevant dissolution media of different pH values.

The pharmacokinetic parameters are shown in Table 26.

TABLE 26
Pharmacokinetic (PK) parameters for Treatment A, B, C and D.

| Parameter | Treatment A | Treatment B | Treatment C | Treatment D |
|---|---|---|---|---|
| Cₘₐₓ (ng/mL) | 59.83 ± 12.91 | 41.54 ± 8.08 | 39.15 ± 7.93 | 49.30 ± 9.26 |
| Tₘₐₓ (h) | 6.1 ± 1.3 | 22.0 ± 11.2 | 33.0 ± 7.7 | 13.7 ± 2.6 |
| AUC₀₋ₜ (ng · h/mL) | 4522 ± 801 | 4478 ± 689 | 4352 ± 752 | 4657 ± 788 |
| AUC_{0-∞} (ng · h/mL) | 4653 ± 830 | 4614 ± 710 | 4484 ± 776 | 4826 ± 839 |
| T_{1/2} (h) | 64.10 ± 10.39 | 63.58 ± 10.10 | 62.66 ± 8.03 | 65.58 ± 13.84 |

TABLE 27
Least-Squares means (90% Confidence Intervals)

| | Treatment B vs. | Treatment C vs. | Treatment D vs. |
|---|---|---|---|
| Parameter | Treatment A | Treatment A | Treatment A |
| Cₘₐₓ (ng/mL) | 69.8 (67.03-72.76) | 65.6 (63.00-68.38) | 82.9 (79.58-86.38) |
| AUC₀₋ₜ (ng · h/mL) | 99.3 (95.43-103.30) | 95.9 (92.18-99.78) | 102.9 (98.88-107.04) |
| AUC_{0-∞} (ng · h/mL) | 99.5 (95.51-103.57) | 96.1 (92.25-100.04) | 103.6 (99.46-107.86) |

Treatments B, C, and D showed an increase in the time of maximum plasma concentration (Tₘₐₓ) following the single dose administration of modified release formulations. A comparison of the area under the plasma concentration (AUC), for modified release formulations to immediate release, showed that all formulations are essentially bioequivalent. The maximum plasma concentration (Cₘₐₓ) versus for treatment B. C and D was significantly reduced as compared to the immediate release dosage form. The AUC values are within 20% of IR tablets suggesting the formulations were equivalent with respect to bio-availability. The Cₘₐₓ reduction was more than 15% for all formulations and was more than 25% for Treatment B and C. Surprisingly, these values are significantly improved from what current In Vitro/In Vivo Correlations (IVIVC) models predicted. One skilled in art will recognize that these models are described in the IVIVC models, based on FDA Guidelines (“Guidance for Industry on Extended Release Oral Dosage Forms: Development, Evaluation, and Application of In Vitro/In Vivo Correlations”, Food and Drug Administration, CDER, September 1997). The terminal half life of all formulations was essentially same which indicates that elimination kinetics was not affected. The AUC values for the modified formulations were within 20% of IR tablets suggesting the formulations were equivalent with respect to bioavailability. As seen in Table 27, the Cₘₐₓ reduction was more than 15% for all formulations and particularly, reduction was more than 25% for Treatment B and C. Surprisingly, these value were significantly improved from IVIVC models predicted used (See Table 28).

TABLE 28
Comparison of pharmacokinetic parameters (dose normalized)

| | Trt B | Trt C | Trt D |
|---|---|---|---|
| Cmax | 41.5 ± 8.08 | 39.2 ± 7.93 | 49.3 ± 7.93 |
| (ng/ml) | | | |
| % Change | −30.6% | 31 34.6 | −17.6 |
| from IR | | | |
| AUC_{0-∞} | 4616 | 4481 | 4818 |
| (ng · h/mL) | | | |
| % Change | −1.1 | −4.0 | 3.2 |
| from IR | | | |
| Tmax (h) | 22.0 | 33.0 | 13.7 |

TABLE 29
Comparison of measured PK parameter versus
IVIVC predicted parameter.

| | Treatment B | Treatment B | Treatment C | Treatment C |
|---|---|---|---|---|
| | Actual | IVIVC Predicted | Actual | IVIVC Predicted |
| | Difference | Difference | Difference | Difference |
| Cmax | −30.6 | −13.9 | −34.6 | −16.2 |
| AUC | −1.1 | −15.7 | −4.0 | −15.7 |

The incidences of adverse effects for the four treatments is shown Table 30. Surprisingly, the modified release formulations of the present invention were better tolerated than the IR tablet (Treatment A). The total AEs were reduced by over 40% for all three treatments.

TABLE 30
Incidence of adverse effects (AEs) from
the Treatments A, B, C and D.

| | Trt A | Trt B | Trt C | Trt D |
|---|---|---|---|---|
| Number of | 16 | 10 | 7 | 10 |
| Subjects with | | | | |
| AEs | | | | |
| Total AEs | 29 | 17 | 12 | 16 |
| Total Dizziness | 13 | 6 | 3 | 5 |
| Events | | | | |
| Number of | 13 | 6 | 3 | 5 |
| Subjects with | | | | |
| Dizziness | | | | |

In terms of adverse events, preliminary data showed that for Treatments A, B, C, and D, the total number of treatment emergent adverse events (TEAEs) was 30, 16, 14, and 17, respectively, indicating a reduction in TEAE observed during treatment with an MR formulation as compared to treatment with the IR tablet. The number of subjects with TEAEs was 18, 11, 7, and 10 for Treatments A, B, C, and D, respectively. The incidence of dizziness for Treatments A, B, C, and D was 14, 7, 4 and 6, respectively.

Treatments B and C met the desired plasma concentration-time profile following single dose administration, while Treatment D does not.

The single 40 mg dose of the prototype MR formulations was better tolerated than the 40 mg IR tablet.

The dosage forms contain excipients that formed less than 3.0% of an adduct formation, preferably less than 2.5%. The adduct formation is detected using HPLC method with an Evaporative Light Scattering Detector.

TABLE 31
Amount of Adduct in the Bead Formulations

| | | (%) Lactose | (%) Other |
|---|---|---|---|
| Stressed Bead Sample | Interval/Conditions | Adduct | Adduct |
| 20% w/w Eudragit ® | 1 mo - 40/75 | None Detected | 0.09 |
| RS/RL (95:5) | | | |
| 20% w/w Eudragit ® | 1 mo - 40/75 | None Detected | 0.07 |
| RS/RL (95:5) | | | |
| 10% w/w Eudragit ® | 6 mo - 40/75 | None Detected | 0.04 |
| RS/RL (95:5) | | | |
| 20% w/w Eudragit ® | 6 mo - 40/75 | None Detected | 0.12 |
| RS/RL (95:5) | | | |
| IR Beads 156.67 mg/g | 6 mo - 40/75 | None Detected | 0.04 |
| 10% w/w Eudragit ® | 3 mo - 40/75 | None Detected | 0.05 |
| RS/RL (94:6) | | | |
| 6% w/w Surelease ® | 1 mo - 40/75 | None Detected | 0.03 |
| (without dessicant) | | | |
| 6% w/w Surelease ® | 1 mo - 40/75 | None Detected | 0.02 |
| (with dessicant) | | | |
| IR Beads 100 mg/g | 36 mo. - ambient | None Detected | 0.02 |
| IR Beads 55 mg/g | 36 mo. - ambient | None Detected | 0.05 |
| IR Beads 55 mg/g | 36 mo. - ambient | None Detected | 0.03 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. An oral dosage form comprising memantine or a salt thereof, wherein the dosage form comprises 2.5 to 100 mg of memantine or a salt thereof and provides an in vivo plasma profile comprising:
a mean Tmax of about 8 or more hours;
a mean Cmax of less than about 100 ng/ml; and
a mean AUC_{0-∞} of more than about 250 ng h/ml.

2. The oral dosage form according to claim 1, wherein the Cmax is less than about 75 ng/ml.

3. The oral dosage form according to claim 1, wherein the Cmax is less than about 50 ng/ml.

4. The oral dosage form according to claim 1, wherein the mean AUC_{0-∞} is more than about 500 ng h/ml.

5. The oral dosage form according to claim 1, wherein the mean AUC_{0-∞} is more than about 1000 ng h/ml.

6. An oral dosage form comprising memantine or a salt thereof, wherein the dosage form comprises 2.5 to 50 mg of memantine or a salt thereof and provides an in vivo plasma profile comprising:
a mean Tmax of about 5 or more hours;
a mean Cmax of less than about 50 ng/ml; and
a mean AUC_{0-∞} of more than about 250 ng h/ml.

7. The oral dosage form according to claim 6, wherein the mean AUC_{0-∞} is more than about 500 ng h/ml.

8. The oral dosage form according to claim 6, wherein the mean AUC_{0-∞} is more than about 1000 ng h/ml.

9. An oral dosage form comprising 2.5 to 100 mg memantine or a salt thereof wherein the dosage form has a dissolution rate of the active ingredient of about 70% to about 80% within about 4 hours to about 24 hours and a Cₘₐₓ of less than about 100 ng/ml, wherein the dosage form provides a reduced incidence of adverse events.

10. An oral dosage form comprising a plurality of beads, wherein each bead comprises:
a core having a diameter from about 1 μm to about 1000 μm and an active ingredient comprising memantine or a salt thereof in the range of about 15 to about 350 mg/g of the dosage form,
wherein the oral dosage form has a dissolution rate of the active ingredient of more than about 80% within about the first 60 minutes following entry of the dosage form into a use environment.

11. The oral dosage form according to claim 10, wherein the dissolution rate of the active ingredient is more than about 80% within about the first 30 minutes following entry of the dosage form into a use environment.

12. The oral dosage form according to claim 10, wherein the dissolution rate of the active ingredient is more than about 80% within about the first 15 minutes following entry of the dosage form into a use environment.

13. The oral dosage form according to claim 10, wherein the active ingredient comprises memantine hydrochloride.

14. The oral dosage form according to claim 10, wherein the dosage form exhibits dose-proportionality.

15. The oral dosage form according to claim 10, wherein the dosage form includes less than about 2.5% adduct

16. The oral dosage form according to claim 10, wherein the active ingredient comprises memantine or a salt thereof in the range of about 15 to about 300 mg/g of the dosage form.

17. The oral dosage form according to claim 10, wherein the active ingredient comprises memantine or a salt thereof in the range of about 25 to about 250 mg/g of the dosage form.

18. The oral dosage form according to claim 10, wherein the core comprise a sugar particle, USP, comprising from about 100 to about 950 mg/g of said dosage form.

19. The oral dosage form according to claim 10, further comprising a glidant in an amount of about 1.5 to about 35 mg/g of said dosage form.

20. The oral dosage form according to claim 19, wherein the glidant is present in an amount from about 5 mg/g to about 30 mg/g.

21. The oral dosage form according to claim 10, wherein the bead dosage forms are compressed into a tablet form.

22. The oral dosage form according to claim 10, further comprising a polymer binder coated on the core.

23. The oral dosage form according to claim 22, wherein the polymer binder is selected from the group consisting of povidone, hydroxypropyl methylcellulose, hydroxypropyl cellulose, and combinations thereof.

24. The oral dosage form according to claim 22, wherein the polymer binder is hydroxypropyl methylcellulose in an amount from about 15 to about 30 mg/g of the dosage form.

25. The oral dosage form according to claim 22, wherein the polymer binder is povidone an amount from about 1.5 to about 35 mg/g of the dosage form.

26. The oral dosage form according to claim 22, further comprising a seal coating applied over the polymer binder.

27. The oral dosage form according to claim 26, wherein the seal coating is selected from the group consisting of HPMC (Opadry®), HPC, Eudragit® RL, Eudragit® E100, Eudragit® E 12.5, Eudragit® E PO, Eudragit® NE, and mixtures thereof.

28. The oral dosage form according to claim 26, wherein the seal coating is present in amounts ranging from about 2% w/w to about 40% w/w.

29. The oral dosage form according to claim 10, wherein the core comprises a sugar sphere or a microcrystalline cellulose sphere in the range of 620-930 mg/g of the dosage form and each bead further comprises:

| Ingredients | Range (mg/g) |
|---|---|
| Povidone, USP | 1.5-35 |
| Talc, USP | 1.5-35 |

30. The oral dosage form according to claim 10, wherein the core comprises a sugar sphere or a microcrystalline cellulose sphere in the range of 700-850 mg/g of the dosage form, the active ingredient comprises memantine or a salt thereof in the range of about 50 to 300 mg/g of the dosage form and wherein each bead further comprises:

| Ingredients | Range (mg/g) |
|---|---|
| Povidone, USP | 5-30 |
| Talc, USP | 5-30 |

31. The oral dosage form according to claim 10, wherein the core comprises a sugar sphere or a microcrystalline cellulose sphere in the range of 500-950 mg/g of the dosage form, the active ingredient comprises memantine or a salt thereof in the range of about 15 to 300 mg/g of the dosage form and wherein each bead further comprises:

| Ingredients | Range (mg/g) |
|---|---|
| Hydroxypropyl Methylcellulose (Opadry) | 15-300 |
| Talc, USP | 1.5-30 |

32. The oral dosage form according to claim 10, wherein the core comprises a sugar sphere or a microcrystalline cellulose sphere in the range of 550-850 mg/g of the dosage form, the active ingredient comprises memantine or a salt thereof in the range of about 25 to 250 mg/g of the dosage form and wherein each bead further comprises:

| Ingredients | Range (mg/g) |
|---|---|
| Hydroxypropyl Methylcellulose (Opadry) | 15-250 |
| Talc, USP | 2.5-25 |

33. An oral dosage form comprising a plurality of beads, each bead comprising
a core having a diameter from about 1 μm to about 1000 μm;
an active ingredient comprising memantine or a salt thereof in the range of about 15 to about 350 mg/g of the dosage form; and
a release modifying polymer layer,
wherein the oral dosage form has a dissolution rate of the active ingredient of about 70% to about 80% within about 4 hours to about 24 hours; and wherein the Cₘₐₓ is less than about 100 ng/ml.

34. The oral dosage form according to claim 33, wherein the dosage form releases the active ingredient for a period of time from about 6 hours to about 48 hours following entry of the dosage form into a use environment.

35. The oral dosage form according to claim 33, wherein the dosage form has a dissolution rate of the active ingredient of about 30% to about 60% within about 2 hours to about 6 hours.

36. The oral dosage form according to claim 33, wherein the dosage form has a dissolution rate of the active ingredient of about 10% to about 50% within about 1 hour.

37. The oral dosage form according to claim 33, wherein the active ingredient comprises memantine hydrochloride.

38. The oral dosage form according to claim 33, wherein the bead dosage forms are compressed into a tablet form.

39. The oral dosage form according to claim 33, wherein the release modifying polymer is selected from the group consisting of ethylcellulose (Surelease®), methacrylate (Eudragit®), methacrylic acid copolymer type C (Acryl-eze®), and mixtures thereof.

40. The oral dosage form according to claim 33, further comprising an intermediate seal coating over the active ingredient.

41. The oral dosage form according to claim 33, further comprising an over coating coated on the release modifying polymer layer.

42. The oral dosage form according to claim 41, wherein the over coating is selected from the group consisting of HPMC (Opadry®), HPC, Eudragit® RL, Eudragit® E100, Eudragit® E 12.5, Eudragit® E PO, Eudragit® NE, and mixtures thereof.

43. The oral dosage form according to claim 33, wherein the dosage form includes less than about 2.5% adduct.

44. The oral dosage form according to claim 33, wherein the core comprises a sugar sphere or a microcrystalline cellulose sphere in the range of 500-900 mg/g of the dosage form, the active ingredient comprises memantine or a salt thereof in the range of about 15 to 350 mg/g of the dosage form and wherein each bead further comprises:

| Ingredients | Range (mg/g) |
|---|---|
| Povidone, USP | 1.5-35 |
| Talc, USP | 1.5-35 |
| Ethylcellulose (Surelease ®) | 100-450 |
| (25% Solid content) | |
| Hydroxypropyl Methylcellulose | 15-30 |
| (Opadry ®) | |

45. The oral dosage form according to claim 33, wherein the core comprises a sugar sphere or a microcrystalline cellulose sphere in the range of 625-800 mg/g of the dosage form, the active ingredient comprises memantine or a salt thereof in the range of about 50 to 285 mg/g of the dosage form and wherein each bead further comprises:

| Ingredients | Range (mg/g) |
|---|---|
| Povidone, USP | 5-30 |
| Talc, USP | 5-30 |
| Ethylcellulose (Surelease ®) | 110-430 |
| (25% Solid content) | |
| Hydroxypropyl Methylcellulose | 15-25 |
| (Opadry ®) | |

46. The oral dosage form according to claim 33, wherein the core comprises a sugar sphere or a microcrystalline cellulose sphere in the range of 580-850 mg/g of the dosage form, the active ingredient comprises memantine or a salt thereof in the range of about 15 to 325 mg/g of the dosage form and wherein each bead further comprises:

| Ingredients | Range (mg/g) |
|---|---|
| Povidone, USP | 1.5-32 |
| Talc, USP | 1.5-32 |
| Ethylcellulose (Surelease ®) (25% | 212-232 |
| Solid content) | |
| HPMC (Opadry ®) | 15-30 |

47. The oral dosage form according to claim 33, wherein the core comprises a sugar sphere or a microcrystalline cellulose sphere in the range of 625-780 mg/g of the dosage form, the active ingredient comprises memantine or a salt thereof in the range of about 30 to 280 mg/g of the dosage form and wherein each bead further comprises:

| Ingredients | Range (mg/g) |
|---|---|
| Povidone, USP | 3-28 |
| Talc, USP | 3-28 |
| Ethylcellulose (Surelease ®) (25% | 212-232 |
| Solid content) | |
| HPMC (Opadry ®) | 15-25 |

48. The oral dosage form according to claim 33, wherein the core comprises a sugar sphere or a microcrystalline cellulose sphere in the range of 625-780 mg/g of the dosage form, the active ingredient comprises memantine or a salt thereof in the range of about 30 to 280 mg/g of the dosage form and wherein each bead further comprises:

| Ingredients | Range (mg/g) |
|---|---|
| Povidone, USP | 3-28 |
| Talc, USP | 3-28 |
| Ethylcellulose (Surelease ®) (25% | 231-430 |
| Solid content) | |
| HPMC (Opadry ®) | 15-25 |

49. The oral dosage form according to claim 33, wherein the core comprises a sugar sphere or a microcrystalline cellulose sphere in the range of 400-750 mg/g of the dosage form, the active ingredient comprises memantine or a salt thereof in the range of about 15 to 300 mg/g of the dosage form and wherein each bead further comprises:

| Ingredients | Range (mg/g) |
|---|---|
| Opadry ® Clear | 15-300 |
| Talc, USP | 1-30 |
| Opadry ® Clear | 10-30 |
| Ammonio Methacrylate Copolymer NF | 75-175 |
| (Eudragit) | |
| Ammonio Methacrylate Copolymer NF | 0.1-26 |
| Type A, (Eudragit) | |
| Triethyl Citrate, NF | 2-30 |
| Talc, USP | 1-70 |
| Opadry ® Clear | 10-30 |

50. The oral dosage form according to claim 33, wherein the core comprises a sugar sphere or a microcrystalline cellulose sphere in the range of 400-750 mg/g of the dosage form, the active ingredient comprises memantine or a salt thereof in the range of about 15 to 300 mg/g of the dosage form and wherein each bead further comprises:

| Ingredients | Range (mg/g) |
|---|---|
| Opadry ® Clear | 15-300 |
| Talc, USP | 1-30 |
| Opadry ® Clear | 10-30 |
| Ammonio Methacrylate Copolymer NF | 235-314 |
| (Eudragit) | |
| Ammonio Methacrylate Copolymer NF | 5-48 |
| Type A, (Eudragit) | |
| Triethyl Citrate, NF | 6-47 |
| Talc, USP | 50-120 |
| Opadry ® Clear | 10-30 |

51. A composite dosage form comprising an immediate release component and a modified release component,
wherein the immediate release component comprises a first plurality of beads, each bead comprising a first active ingredient comprising memantine or a salt thereof in the range of about 15 to about 350 mg/g of the dosage form, wherein about 80% of the first active ingredient dissolves within about the first 60 minutes following entry of the dosage form into a use environment; and
wherein the modified release component comprises a second plurality of beads, each bead comprising a second active ingredient comprising memantine or a salt thereof in the range of about 15 to about 350 mg/g of the dosage form, wherein about 70% to about 80% of the second active ingredient dissolves within about 4 hours to about 24 hours following entry of the dosage form into the use environment.

52. The composite dosage form of claim 51, wherein the composite dosage form is compressed into a tablet form.

53. The composite dosage form of claim 51, wherein the composite dosage form comprises from about 2.5 mg to about 100 mg of memantine.

54. A method for treating a condition selected from the group selected from Alzheimer's disease, autism and neuropathic pain comprising administering to a patient in need thereof the oral dosage form of claim 1.

55. A method for treating a condition selected from the group selected from Alzheimer's disease, autism and neuropathic pain comprising administering to a patient in need thereof the oral dosage form of claim 6.

56. A method for treating a condition selected from the group selected from Alzheimer's disease, autism and neuropathic pain comprising administering to a patient in need thereof the oral dosage form of claim 9.

57. A method for treating a condition selected from the group selected from Alzheimer's disease, autism and neuropathic pain comprising administering to a patient in need thereof the oral dosage form of claim 10.

58. A method for treating a condition selected from the group selected from Alzheimer's disease, autism and neuropathic pain comprising administering to a patient in need thereof the oral dosage form of claim 33.

59. A method for treating a condition selected from the group selected from Alzheimer's disease, autism and neuropathic pain comprising administering to a patient in need thereof the oral dosage form of claim 51.
